(19) United States
(12) Patent Application Publication
(10) Pub. No.: US 2007/0092527 A1
(43) Pub. Date: Apr. 26, 2007

(54) Proton-sensing g-protein coupled receptors and dna sequences thereof

(75) Inventors: Birgit Baumgarten, Inzlingen (DE); Carol Elizabeth Jones, Horsham (GB); Marie-Gabrielle Ludwig, Michelbach-le-bas (FR); Georg Martiny-Baron, Herbolzheim (DE); Klaus Seuwen, Michelbach le Bas (FR); Romain Wolf, Schlierbach (FR); Lorenza Wyder, Basel (CH); Thomas Suply, Zimmersheim (FR)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 10/560,322

(22) PCT Filed: Jun. 18, 2004
(86) PCT No.: PCT/EP04/06625

§ 371 Date: Sep. 12, 2006

Related U.S. Application Data
(60) Provisional application No. 60/480,245, filed on Jun. 20, 2003.
(60) Provisional application No. 60/499,549, filed on Sep. 2, 2003.

(51) Int. Cl. G01N 33/567; A61K 39/00; A61K 38/17

(52) U.S. Cl. 424/185.1; 514/12; 435/7.2

(57) ABSTRACT

This invention relates to the newly identified utility of certain G protein-coupled receptors (GPCRs), in particular to OGR1, GPR4 and TDAG8 (TDAG8 is also named GPR65) polypeptides, and polynucleotides encoding such GPCR polypeptides, to their use in diagnosis and to methods of identifying compounds that are agonists or antagonists to said GPCRs, and to the production of such polypeptides and polynucleotides.

### FIELD OF THE INVENTION

This invention relates to the newly identified uses of proton-sensing G protein-coupled receptor (herein after referred to as “proton-sensing GPCRs”) polypeptides and polynucleotides encoding such polypeptides, to their use in diagnosis and in identifying compounds that are agonists, antagonists of such proton-sensing GPCRs, and to the production of such polypeptides and polynucleotides.

### BACKGROUND OF THE INVENTION

In addition to its role in calcium metabolism, bone also plays a major role in regulating pH homeostasis. Bone has considerable buffering capacity that can be mobilized immediately (chemical equilibria) and slowly, by cell-mediated processes (Bushinsky D A, 2001, Eur J Nutr, 40: 238-244). In chronic acidosis, bone resorption is increased; in contrast alkalosis tends to stimulate bone formation (Lemann J, et al., 1966, J Clin Invest, 45: 1608-1614; Amett T R, et al., 1996, Bone, 18: 277-279; Bushinsky DA, 1996, Am J Physiol, 271:F216-222; Bushinsky DA, 1999, Am J Physiol, 277: F813-819). Due to suboptimal nutrition and declining renal function elderly people often present with a mild chronic acidosis, that may lead to increased bone resorption, and hence participate in the development of osteoporosis (Bushinsky D A, 2001, Eur J Nutr, 40: 238-244; Frassetto L A, et al., 1996, Am J Physiol, 271: F1114-22). The pH-sensing mechanisms operating in bone cells are as yet unknown.

Airway concentrations of many reactive nitrogen and oxygen species are high in asthma. The stability and bioactivities of these species are pH-dependent. The pH of dearated exhaled airway vapor condensate is over two log orders lower in patients with acute asthma than in control subjects and normalizes with corticosteroid therapy suggesting that airway pH is an important determinant of airway inflammation (Hunt J F, et al., 2000, Am J Resp, 161: 694-699). Airway acidity has been shown to accelerate eosinophil necrosis also suggesting a role in airway inflammation (Hunt J F, et al., 2000, Am J Resp, 161: 694-699). Again, the pH-sensing mechanisms of cells in the airway are unknown. Furthermore, It was found recently that extracellular activation of neutrophils induces their activation (Trevani A S, et al., 1999, J Immunology, 162: 4849-4857). Neutrophils play an important role in host defense against infectious agents and are involved In the pathogenesis of a plethora of inflammatory conditions (Ganz T M, et al., 1988, Ann Intern Med. 109:127). Reported effects of extracellular acidosis on the delay of spontaneous neutrophil apoptosis, extending the neutrophil functional lifespan also suggest a role of acidification in airway inflammation (Trevani A S, et al., 1999, J. Immunol, 162:4849-4857). Other evidence to support a role of acidification in airway inflammation include the reduction or elimination of cilia beat frequency In human bronchial epithelial explants at reduced pH (<6.5) (Luk K A, et al., 1983, Clin Sci, 64:449-451), the effects of reduced pH on respiratory mucus viscosity (Holma B O, 1985, Sci Total Environ, 41:101-123) and the effect of airway acidification on cough in a guinea pig model (Ricciardo F L M, et al., 1999, Am J Resp Crit Care Med, 159:557-562).

Acidosis is a hallmark of a variety of diseases such as tumors (Wike-Hooley et al., Radiother Oncol 1984, 2: 343-66) and Ischemia (Webster K A, Cardivasc. Toxicol. 2003, 3: 283-98) where angiogenesis is known to play a key role. However, the effect of acidosis on endothelial cells is not well understood. Recently, it has been shown that acidic extracellular pH can protect endothelial cells from apoptosis (Terminelia et al., Am. J. Physiol lung Cell Mol Physiol 2002, 283: L1291-302). In addition, acidosis inhibited endothelial cell proliferation, migration as well as capillary formation stimulated by 10% FCS, this despite the production and presence of enhanced amounts of VEGF or bFGF (D'Arcangelo et al., Circ. Res. 2000, 86: 312-8). In presence of acidic extracellular pH a marked delay in microvascular growth from aortic rings cultured in 3 dimensional collagen gels could be observed. This delay was reduced in the presence of exogenous growth factors such as VEGF and bFGF indicating that in vivo angiogenic responses could still occur since in those cases high amounts of angiogenic growth factors are present (Burbridge et al., Angiogenesis 1999, 3: 281-88).

In tumors rather than being detrimental acidic environment has been shown to be conductive to tumor progression, inducing migration and invasion of tumor cells (Martinez-Zaguilan et al, Clin Exp. Metastasis 1996, 14, 176-86), the secretion of matrix-degrading metalloproteases (MMPs) (Kato et al, Cell Biol. Intn 1996, 20.375-7). as well as the production of angiogenic factors such as vascular endothelial growth factor (VEGF) (Shi et al., Oncogene 2001, 20:3751-6) and IL8 (Shi et al., Clin Cancer Res. 1999, %: 3711-21; Karashima et al Clin Cancer Res 2003, 9: 2786-97) from tumor cells both in vitro and in vivo (Fukumura et al., Cancer Res. 2001, 61: 6020-4).

GPR4 (herein identified as a pH sensor) has been shown to be expressed in HUVECs (human umbilical vein endothelial cells) and siRNA against GPR4 showed impaired proliferation and tube formation of HUVECs (Xu Y et al., 2003, First annual Atherothrombosis Summit: Arterial Inflammation (September. 17-19), abstract 5).

### SUMMARY OF THE INVENTION

The present invention is based on our surprising discovery that certain G protein-coupled receptors, in particular OGR1, GPR4 and TDAG8 (TDAG8 is also named GPR65), act as proton-sensing receptors (proton-sensing GPCRs). Thus, the present invention relates to the novel use of certain GPCRs with proton-sensing functionality, polynucleotides encoding such polypeptides, recombinant materials and methods for their production. Such polypeptides and polynucleotides are of interest in relation to methods of treatment of certain diseases, including, but not limited to diseases and medical conditions in which proton homeostasis is altered, e.g. in diseases and medical conditions involving elevated levels of protons, i.e. hydrogen ion, including diseases of excessive bone loss, including osteoporosis, especially senile osteoporosis and osteoporosis due to renal failure. Besides bone metabolism proton-sensing GPCRs may be involved in the regulation of respiration and cardiovascular functions and pathological states linked to detonation of the blood supply, like inflammation and ischemia.

In a further aspect, the invention relates to methods for identifying agonists and antagonists (e.g., inhibitors) of proton-sensing GPCRs using the genes and polypeptides provided by the invention, and treating conditions associated with a proton imbalance with the identified compounds. In a still further aspect, the invention relates to diagnostic assays for detecting diseases associated with inappropriate proton-sensing GPCRs activity, activities or levels.

### DESCRIPTION OF THE INVENTION

In a first aspect, the present invention relates to a novel use of certain GPCR polypeptides in pH homeostasis.

Such polypeptides are selected from one of the groups consisting of:

(a) an isolated polypeptide encoded by a polynucleotide comprising the polynucleotide sequence of human OGR1 (accession number NM_{—}003485.1), rat OGR1 (accession number: XM_{—}234483), mouse OGR1 (accession number: NM_{—}175493), bovine OGR1 (accession number: NM_{—}174329), preferably human OGR1 (accession number. NM_{—}003485.1), human GPR4 (accession number: NM_{—}005282), mouse GPR4 (accession number: NM_{—}175668), human TDAG8 (accession number: NM_{—}003608) and mouse TDAG8 (accession number NM_{—}008152);

(b) an Isolated proton sensing GPCR polypeptide comprising a polypeptide sequence having at least 80%, preferably 85%, more preferably 90%, more preferably 95%, more preferably 96%, more preferably 97%, more preferably 98%, or more preferably 99% identity to the polypeptide sequence of SEQ ID NO: 1;

(c) an isolated proton sensing GPCR polypeptide comprising a polypeptide sequence having at least 20%, preferably 30%, more preferably 32%, more preferably 35%, more preferably 45% to the polypeptide sequence of SEQ ID NO: 1;

(d) an isolated proton sensing GPCR polypeptide comprising a polypeptide sequence having at least 80%, preferably 85%, more preferably 90%, more preferably 95%, more preferably 96%, more preferably 97%, more preferably 98%, or more preferably 99% identity to the polypeptide sequence of SEQ ID NO: 3;

(d) an isolated proton sensing GPCR polypeptide comprising a polypeptide sequence having at least 20%, preferably 30%, more preferably 32%, more preferably 35%, more preferably 45% to the polypeptide sequence of SEQ ID NO: 3;

(e) an isolated proton sensing GPCR polypeptide comprising a polypeptide sequence having at least 80%, preferably 85%, more preferably 90%, more preferably 95%, more preferably 96%, more preferably 97%, more preferably 98%, or more preferably 99% identity to the polypeptide sequence of SEQ ID NO: 4;

(f) an isolated proton sensing GPCR polypeptide comprising a polypeptide sequence having at least 20%, preferably 30%, more preferably 32%, more preferably 35%, more preferably 45% identity to the polypeptide sequence of SEQ ID NO: 4;

(g) an isolated polypeptide comprising the polypeptide sequence of SEQ ID NO: 1, SEQ ID NO: 3 or SEQ ID NO: 4;

(h) an isolated proton sensing GPCR polypeptide having at least 80%, preferably 85%, more preferably 90%, more preferably 95%, more preferably 96%, more preferably 97%, more preferably 98%, or more preferably 99% identity to the polypeptide sequence of SEQ ID NO: 1;

(i) an Isolated proton sensing GPCR polypeptide having at least 20%, preferably 30%, more preferably 32%, more preferably 35%, more preferably 45% identity to the polypeptide sequence of SEQ ID NO: 1;

(k) an isolated proton sensing GPCR polypeptide having at least 80%, preferably 85%, more preferably 90%, more preferably 95%, more preferably 96%, more preferably 97%, more preferably 98%, or more preferably 99% identity to the polypeptide sequence of SEQ ID NO: 3;

(l) an isolated proton sensing GPCR polypeptide having at least 20%, preferably 30%, more preferably 32%, more preferably 35%, more preferably 45% identity to the polypeptide sequence of SEQ ID NO: 3;

(m) an isolated proton sensing GPCR polypeptide having at least 80%, preferably 85%, more preferably 90%, more preferably 95%, more preferably 96%, more preferably 97%, more preferably 98%, or more preferably 99% identity to the polypeptide sequence of SEQ ID NO: 4;

(n) an isolated proton sensing GPCR polypeptide having at least 20%, preferably 30%, more preferably 32%, more preferably 35%, more preferably 45% Identity to the polypeptide sequence of SEQ ID NO: 4;

(o) the polypeptide sequences of SEQ ID NO: 1, SEQ ID NO: 3 or SEQ ID NO: 4;

(p) an isolated proton sensing GPCR polypeptide having or comprising a polypeptide sequence that has an Identity Index of 0.20, preferably 0.30, more preferably 0.32, more preferably 0.35, more preferably 0.45, compared to the polypeptide sequence of SEQ ID NO: 1, SEQ ID NO: 3 or SEQ ID NO: 4;

(q) fragments and variants of such polypeptides in (a) to (p); and

(r) polypeptides in (a) to (p) which show a pH-dependent Inositol phosphate or cAMP formation in CCL39 hamster fibroblast cells or a pH-dependent signal in the cAMP luciferase reporter assay in CHOK1 CRE-luc cells or CCL39 CRE-luc cells.

Polypeptides of the present invention are members of the G protein-coupled receptors family of polypeptides. The biological properties of the proton-sensing GPCRs polypeptides as defined herein (e.g. linked to onset of osteoporosis, respiratory diseases, e.g. asthma, acute/adult respiratory distress syndrome (ARDS), chronic obstructive pulmonary (COPD), and cardiovascular diseases) are hereinafter referred to as “biological activity or activities of the proton-sensing GPCRs” or “proton-sensing activity”. Preferably, a polypeptide of the present invention exhibits at least one biological activity of the proton-sensing GPCRs as defined above. More preferably, a polypeptide of the present invention exhibits at least one biological activity of OGR1, GPR4 or TDAG8. E.g. the primary biological property of human OGR1 polypeptides and human TDAG8 polypeptides are linked to bone-resorbing diseases, including but not limited to diseases with excessive bone loss, including osteoporosis, gingival diseases such as gingivitis and periodontitis, Paget's disease, hypercalcemia of malignancy, e.g. tumour-Induced hypercalcemia and metabolic bone disease. In addition, the primary biological property of human GPR4 is e.g. linked to diseases (modifying compounds, i.e. compounds with agonistic or/and antagonistic action may be helpful in those diseases) which are implicated in angiogenesis, resulting, for example, cancer diseases, e.g. treatment of solid tumors, diseases of the heart, e.g. cardiovascular diseases such as myocardial infarction, limb diseases, e.g. peripheral arterial occlusive disease, eye diseases, e.g. diabetic retinopathy or macular degeneration, arthritis, e.g. rheumatoid arthritis, diseases where wound care is important, and diseases of the skin. Moreover, GPR4 is e.g. linked to diseases which are implicated in inflammatory or obstructive airways diseases, resulting, for example, in reduction of tissue damage, bronchial hyperreactivity, remodelling or disease progression; said inflammatory or obstructive airways diseases include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection.

Polypeptides of the present invention also includes variants of the aforementioned polypeptides, including all allelic forms and splice variants. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from to 5, from 5 to 3, from 3 to 2, from 2 to 1 or 1 amino acids are inserted, substituted, or deleted, in any combination.

Preferred fragments of polypeptides of the present invention include an isolated polypeptide comprising an amino acid sequence having at least 30, 50 or 100 contiguous amino acids from the amino acid sequence of SEQ ID NO: 1, SEQ ID NO: 3, SEQ ID NO: 4 or an isolated polypeptide comprising an amino acid sequence having at least 30, 50 or 100 contiguous amino acids truncated or deleted from the amino acid sequence of SEQ ID NO: 1, SEQ ID NO: 3, SEQ ID NO: 4. Preferred fragments are biologically active fragments that block or enhance the biological activity of GPCRs of the invention, in particular OGR1, GPR4 or TDAG8, including those with a similar activity or an improved activity, or with a decreased undesirable activity. Also preferred are those fragments that are antigenic or immunogenic In an animal, especially In a human.

Fragments of the polypeptides of the invention may be employed for producing the corresponding full-length polypeptide by peptide synthesis; therefore, these variants may be employed as intermediates for producing the full-length polypeptides of the Invention. The polypeptides of the present invention may be in the form of the “mature” protein or maybe a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. The means for preparing such polypeptides are well understood in the art.

In a further aspect, the present invention relates to a novel use of a proton sensing GPCR polynucleotide in pH homeostasis. Such a polynucleotide is selected from one of the groups consisting of:

(a) an isolated polynucleotide comprising the polynucleotide sequence of human OGR1 (accession number. NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number: NM_{—}008152), preferably human OGR1, human TDAG8 and human GPR4;

(b) an isolated polynucleotide encoding a proton sensing GPCR polypeptide sequence having at least 80%, preferably 85%, more preferably 90%, more preferably 95%, more preferably 96%, more preferably 97%, more preferably 98%, or more preferably 99% Identity to the polypeptide sequence of SEQ ID NO: 1;

(c) an isolated polynucleotide encoding a proton sensing GPCR polypeptide sequence having at least 20%, preferably 30%, more preferably 32%, more preferably 35%, more preferably 45% to the polypeptide sequence of SEQ ID NO: 1;

(d) an isolated polynucleotide encoding a proton sensing GPCR polypeptide sequence having at least 80%, preferably 85%, more preferably 90%, more preferably 95%, more preferably 96%, more preferably 97%, more preferably 98%, or more preferably 99% identity to the polypeptide sequence of SEQ ID NO: 3;

(d) an isolated polynucleotide encoding a proton sensing GPCR polypeptide sequence having at least 20%, preferably 30%, more preferably 32%, more preferably 35%, more preferably 45% to the polypeptide sequence of SEQ ID NO: 3;

(e) an Isolated polynucleotide encoding a proton sensing GPCR polypeptide sequence having at least 80%, preferably 85%, more preferably 90%, more preferably 95%, more preferably 96%, more preferably 97%, more preferably 98%, or more preferably 99% identity to the polypeptide sequence of SEQ ID NO: 4;

(f) an Isolated polynucleotide encoding a proton sensing GPCR polypeptide sequence having at least 20%, preferably 30%, more preferably 32%, more preferably 35%, more preferably 45% identity to the polypeptide sequence of SEQ ID NO: 4;

(g) an isolated polynucleotide comprising the polynucleotide sequence of human OGR1 (accession: NM_{—}003485.1), rat OGR1 (accession: XM_{—}234483), mouse OGR1 (accession: NM_{—}175493), bovine OGR1 (accession: NM_{—}174329), human GPR4 (accession: NM_{—}005282);

(h) the polynucleotide sequences of human OGR1 (accession: NM_{—}003485.1), rat OGR1 (accession: XM_{—}234483), mouse OGR1 (accession: NM_{—}175493), bovine OGR1 (accession: NM_{—}174329), human GPR4 (accession: NM_{—}005282);

(i) an isolated polynucleotide encoding a polypeptide sequence that has an Identity Index of 0.20, preferably 0.30, more preferably 0.32, more preferably 0.35, more preferably 0.45, compared to the polypeptide sequence of SEQ ID NO: 1, SEQ ID NO: 3 or SEQ ID NO: 4;

(q) fragments and variants of such polynucleotides in (a) to (i); and

(r) polynucleotides in (a) to (l) which encode for a polypeptide that show a pH-dependent Inositol phosphate or cAMP formation In CCL39 hamster fibroblast cells or a pH-dependent signal In the cAMP luciferase reporter assay in CHOK1 CRE-luc cells or CCL39 CRE-luc cells.

Preferred fragments of polynucleotides for use in modulating pH homeostasis include an isolated polynucleotide comprising an nucleotide sequence having at least 15, 30, 50 or 100 contiguous nucleotides from the sequence of human OGR1 (accession number: NM_{—}003485.1), rat OGR1 (accession number: XM_{—}234483), mouse OGR1 (accession number NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number: NM_{—}008152) or an isolated polynucleotide comprising a sequence having at least 30, 50 or 100 contiguous nucleotides truncated or deleted from the sequence of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number: NM_{—}008152).

Preferred variants of polynucleotides for use in modulating pH homeostasis include splice variants, allelic variants, and polymorphisms, including polynucleotides having one or more single nucleotide polymorphisms (SNPs).

Polynucleotides for use in modulating pH homeostasis also include polynucleotides encoding polypeptide variants that comprise the amino acid sequence of SEQ ID NO: 1, SEQ ID NO: 3 or SEQ ID NO: 4 and in which several, for instance from 50 to 30, from 30 to 20, from 20 to 10, from 10 to 5, from 5 to 3, from 3 to 2, from 2 to 1 or 1 amino acid residues are substituted, deleted or added, in any combination.

In a further aspect, the present invention provides polynucleotides for use in modulating pH homeostasis that are RNA transcripts of the DNA sequences of the present invention. Accordingly, there is provided an RNA polynucleotide for use in modulating pH homeostasis that:

(a) comprises an RNA transcript of the DNA sequence encoding the polypeptide of SEQ ID NO: 1, SEQ ID NO: 3 or SEQ ID NO: 4;

(b) is the RNA transcript of the DNA sequence encoding the polypeptide of SEQ ID NO: 1, SEQ ID NO: 3 or SEQ ID NO: 4;

(c) comprises an RNA transcript of the DNA sequences of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (NM_{—}008152); or

(d) is the RNA transcript of the DNA sequence of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number: NM_{—}008152) and RNA polynucleotides that are complementary thereto.

The polynucleotide sequence of human OGR1 (accession number: NM_{—}003485.1) is a cDNA sequence that encodes the polypeptide of SEQ ID NO: 1. The polynucleotide sequence encoding the polypeptide of SEQ ID NO: 1 may be identical to the polypeptide encoding sequence of human OGR1 (accession number: NM_{—}003485.1) or it may be a sequence other than human OGR1 (accession number: NM_{—}003485.1) which, as a result of the redundancy (degeneracy) of the genetic code, also encodes the polypeptide of SEQ ID NO: 1.

The polynucleotide sequence of human GPR4 (accession number: NM_{—}005282) is a cDNA sequence that encodes the polypeptide of SEQ ID NO: 3. The polynucleotide sequence encoding the polypeptide of SEQ ID NO: 3 may be identical to the polypeptide encoding sequence of human GPR4 (accession number: NM_{—}005282) or it may be a sequence other than human GPR4 (accession number: NM_{—}005282) which, as a result of the redundancy (degeneracy) of the genetic code, also encodes the polypeptide of SEQ ID NO: 3.

The polynucleotide sequence of human TDAG8 (accession number: NM_{—}003608) is a cDNA sequence that encodes the polypeptide of SEQ ID NO: 4. The polynucleotide sequence encoding the polypeptide of SEQ ID NO: 4 may be identical to the polypeptide encoding sequence of human TDAG8 (accession number: NM_{—}003608) or it may be a sequence other than human TDAG8 (accession number: NM_{—}003608) which, as a result of the redundancy (degeneracy) of the genetic code, also encodes the polypeptide of SEQ ID NO: 4.

Polynucleotides for use in modulating pH homeostasis may be obtained using standard cloning and screening techniques from a cDNA library derived from mRNA in e.g. brain, kidney, lung and cells of the immune system (for expression of OGR1 see e.g. Xu Y, et al., 2000, Nat Cell Biol, 2:261-267; Zhu K, et al., 2001, J Biol Chem, 276:41325-41335; Xu Y, et al., 1996, Genomics, 35:397-402; for expression of GPR4 see An S, et al., 1995, FEBS Letts, 375:121-124; for expression of TDAG8 see Kyaw H, et al., 1998, DNA Cell Biol, 17:493-500 and Choi J W, et al., 1996, Cell Immunol, 168:78-84.) (for standard cloning technique see for instance, Sambrook J, et al., 1989, Molecular Cloning: A Laboratory Manual, 2nd Ed., Cold Spring Harbor Laboratory Press, Cold Spring Harbor, N.Y.). Polynucleotides of the invention can also be obtained from natural sources such as genomic DNA libraries or can be synthesized using well known and commercially available techniques.

When polynucleotides of the present invention are used for the recombinant production of polypeptides for use in modulating pH homeostasis, the polynucleotide may include the coding sequence for the mature polypeptide, by itself, or the coding sequence for the mature polypeptide In reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz R, et al., 1989, Proc Natl Acad Sci USA 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

A polynucleotide encoding a polypeptide of the present invention, including homologs from species not yet known, may be obtained by a process comprising the steps of screening a library under stringent hybridization conditions with a labelled probe having the sequence of SEQ ID NO: 1, SEQ ID NO: 3 or SEQ ID NO: 4, or a fragment thereof, preferably of at least 15 nucleotides; and isolating full-length cDNA and genomic clones containing said polynucleotide sequence. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides for use in modulating pH homeostasis, preferably with a nucleotide sequence of at least 100, obtained by screening a library under stringent hybridization conditions with a labeled probe which is complementary, i.e. hybridize to the polynucleotide encoding for the sequence of SEQ ID NO: 1, SEQ ID NO: 3 or SEQ ID NO: 4 or a fragment thereof, preferably of at least 15 nucleotides.

The person skilled in the art will appreciate that, in many cases, an isolated cDNA sequence will be incomplete, in that the region coding for the polypeptide does not extend all the way through to the 5′ terminus. This is a consequence of reverse transcriptase, an enzyme with inherently low “processivity” (a measure of the ability of the enzyme to remain attached to the template during the polymerisation reaction), failing to complete a DNA copy of the mRNA template during first strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman M A, et al., 1988, Proc Nat Acad Sci USA, 85:8998-9002). Recent modifications of the technique, exemplified by the Marathon (trade mark) technology (Clontech Laboratories, Inc.) for example, have significantly simplified the search for longer cDNAs. In the Marathon (trade mark) technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analysed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., 1986, Basic Methods in Molecular Biology and Sambrook J, et al. (ibid).

Preferred methods of introducing polynucleotides into host cells include, for instance, calcium phosphate transfection, DEAE-dextran mediated transfection, transfection, microinjection, cationic lipid-mediated transfection, electroporation, transduction, scrape loading, ballistic introduction or infection.

Representative examples of appropriate hosts include bacterial cells, such as Streptococci, Staphylococci, E coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook J, et al. (see above). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and/or purification.

Polynucleotides of the present invention may be used as diagnostic reagents, through detecting mutations in the associated gene. Detection of a mutated form of the gene characterised by the polynucleotide of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number NM_{—}005282), mouse GPR4 (accession number: NM_{—}175668), human TDAG8 (accession number: NM_{—}003608) and mouse TDAG8 (accession number: NM_{—}008152) in the cDNA or genomic sequence and which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled OGR1 nucleotide sequences. Perfectly matched sequences can be distinguished from mismatched duplexes by RNase digestion or by differences in melting temperatures.

DNA sequence difference may also be detected by alterations in the electrophoretic mobility of DNA fragments in gels, with or without denaturing agents, or by direct DNA sequencing (see, for instance, Myers R M, et al., 1985, Science, 230:1242-1246). Sequence changes at specific locations may also be revealed by nuclease protection assays, such as RNase and S 1 protection or the chemical cleavage method (see Cotton et al., 1985, Proc Natl Acad Sci USA, 85:4397-4401).

An array of oligonucleotides probes comprising polynucleotides of the present invention or fragments thereof can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, Chee M, et al., 1996, Science, 274:610-613 and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for Instance PCR, RT-PCR, RNase protection, Northern blotting and other hybridization methods. Assay techniques that can be used to determine levels of a protein, such as a polypeptide of the present invention, in a sample derived from a host are well-known to those skilled In the art. Such assay methods include radioimmunoassays, competitive-binding assays, Western Blot analysis and ELISA assays.

Thus in another aspect, the present invention relates to a diagonostic kit comprising:

(a) a polynucleotide of the present invention, preferably the nucleotide sequence of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number: NM_{—}008152) or a fragment or an RNA transcript thereof;

(b) a nucleotide sequence complementary to that of (a);

(c) a polypeptide of the present invention, preferably the polypeptide of SEQ ID NO: 1, SEQ ID NO: 3, SEQ ID NO: 4 or a fragment thereof; or

(d) an antibody to a polypeptide of the present invention, preferably to the polypeptide of SEQ ID NO: 1, SEQ ID NO: 3, SEQ ID NO: 4.

It will be appreciated that in any such kit, (a), (b), (c) or (d) may comprise a substantial component. Such a kit will be of use in diagnosing a disease or susceptibility to a disease, particularly diseases of the invention, amongst others.

The polypeptides of the present invention are expressed in e.g. brain, kidney, lung and cells of the immune system (Xu Y, et al., 2000, Nat Cell Biol, 2:261-267; Zhu K, et al., 2001, J Biol Chem, 276:41325-41335; Xu Y, et al., 1996, Genomics, 35:397-402).

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art. Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler G and Milstein C, 1975, Nature, 256:495-497), the trioma technique, the human B-cell hybridoma technique (Kozbor D, et al., 1983, Immunology Today, 4:72) and the EBV-hybridoma technique (Cole et al., 1985, Monoclonal Antibodies and Cancer Therapy, 77-96, Alan R. Liss, Inc.).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An Immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down-in the stomach, it is preferably administered parenterally (for instance, subcutaneous, intramuscular, intravenous, or intradermal injection). Formulations suitable for parenteral administration include aqueous and non-aqueous sterile injection solutions that may contain anti-oxidants, buffers, bacteriostats and solutes that render the formulation isotonic with the blood of the recipient; and aqueous and non-aqueous sterile suspensions that may include suspending agents or thickening agents.

The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in the prevention and treatment of one or more disease states. Such disease states are diseases the protein level or the activity of the polypeptides of the invention are abnormal, i.e. not are within the normal range. Such diseases include e.g. diseases involving infection by organisms such as pneumocystis carinii, trypsanoma cruzi, trypsanoma brucei, crithidia fusiculata, as well as parasitic diseases such as schistosomiasis and malaria, tumours (tumour invasion and tumour metastasis), and other diseases such as metachromatic leukodystrophy, muscular dystrophy, amytrophy and similar diseases. Furthermore, polypeptides of the invention may be implicated in diseases with excessive bone loss, including osteoporosis, gingival diseases such as gingivitis and periodontitis, Paget's disease, hypercalcemia of malignancy, e.g. tumour-induced hypercalcemia and metabolic bone disease. Furthermore, polypeptides of the invention may be implicated in diseases of excessive cartilage or matrix degradation, including osteoarthritis and rheumatoid arthritis as well as certain neoplastic diseases involving expression of high levels of proteolytic enzymes and matrix degradation. Furthermore, polypeptides of the invention may be implicated in coronary disease, including atherosclerosis (including atherosclerotic plaque rupture and destabilization), autoimmune diseases, respiratory diseases and immunologically mediated diseases (including transplant rejection). Furthermore, polypeptides of the invention may be implicated in osteoporosis of various genesis (e.g. juvenile, menopausal, post-menopausal, post-traumatic, caused by old age or by cortico-steroid therapy or inactivity).

Moreover, polypeptides of the invention may be implicated in inflammatory or obstructive airways diseases, resulting, for example, in reduction of tissue damage, bronchial hyperreactivity, remodelling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Subjects with asthma also include subjects, e.g. of less than 4 or 5 years of age, exhibiting wheezing symptoms and diagnosed or diagnosable as “wheezy infants”, an established patient category of major medical concern and now often identified as incipient or early-phase asthmatics (For convenience this particular asthmatic condition is referred to as “wheezy-infant syndrome”). Furthermore, polypeptides of the invention may be implicated in prophylactic treatment of asthma and this will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy. Furthermore, polypeptides of the invention may be implicated in other inflammatory or obstructive airways diseases and conditions, such as acute lung injury (ALI), acute/adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The polypeptides of the invention are also related to bronchitis of whatever type or genesis Including, e.g., acute, arachidic, catarrhal, croupus, chronic or phthinoid bronchitis. Further inflammatory or obstructive airways diseases to which the present polypeptides of the invention are related include pneumoconlosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis. Having regard to their anti-inflammatory activity, in particular in relation to inhibition of eosinophil activation, the polypeptides of the invention are also related to disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Beneficial effects are evaluated in in vitro and in vivo pharmacological tests generally known in the art, and as illustrated herein. The above cited properties are demonstrable in in vitro and in vivo tests, using advantageously mammals, e.g. rats, mice, dogs, rabbits, monkeys or isolated organs and tissues, as well as mammalian enzyme preparations, either natural or prepared by e.g. recombinant technology. Agonists or antagonists to the polypeptides of the invention, which can be obtained by screening assays as described herein, e.g. in the Example 10, can be applied in vitro in the form of solutions, e.g. preferably aqueous solutions or suspensions, and in vivo either enterally or parenterally, advantageously orally, e.g. as a suspension or in aqueous solution, or as a solid capsule or tablet formulation. In case of asthma or similar diseases, the delivery of agonists and antagonists may be done directly to the lung. The dosage in vitro may range between about 10⁻⁵ molar and 10⁻⁹ molar concentrations. The dosage in vivo may range, depending on the route of administration, between about 0.1 and 100 mg/kg.

Screening techniques: The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

A polypeptide of the present invention may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide that compete with the binding of the polypeptide to its receptors, if any. Standard methods for conducting such assays are well understood in the art.

Examples of antagonists of polypeptides of the present invention include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or a small molecule that bind to the polypeptide of the present invention but do not elicit a response, so that the activity of the polypeptide is prevented.

Screening methods may also involve the use of transgenic technology. The art of constructing transgenic animals is well established. For example, the OGR1, GPR4 or TDAG8 gene may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention.

Screening kits for use in the above described methods form a further aspect of the present invention. Such screening kits comprise:

(a) a polypeptide of the present invention;

(b) a recombinant cell expressing a polypeptide of the present invention,

(c) a cell membrane expressing a polypeptide of the present invention; or

(d) an antibody to a polypeptide of the present invention; which polypeptide is preferably that of SEQ ID NO: 1, SEQ ID NO: 3 or SEQ ID NO: 4;

It will be appreciated that in any such kit, (a), (b), (c) or (d) may comprise a substantial component.

Glossary

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered by the human hands from its natural state, i.e. if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons.

“Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, —i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutarnate, formylation, gamma-carboxylation, glycosylation, GPI anchor formation, hydroxylation, iodination, methylation, myristoylation, oxidation, proteolytic processing, phosphorylation, prenylation, racernization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide of SEQ ID NO: 1, SEQ ID NO: 3 or SEQ ID NO: 4. “Fragment” of a polynucleotide sequence refers to a polynucleotide sequence that is shorter than the reference sequence of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number NM_{—}175668), human TDAG8 (accession number: NM_{—}003608) and mouse TDAG8 (accession number: NM_{—}008152).

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gin, Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being—compared (so-called global alignment), that is particularly suitable for sequences of the same or very similar length, or over shorter, defined lengths (so-called local alignment), that is more suitable for sequences of unequal length.

“Imbalanced pH range/imbalanced proton homeostasis”—The range of normal arterial pH (7.36 to 7.44) encompasses approximately two standard deviations of the normal population; anything outside this range is considered abnormal. Clinically, the “safe” range for pH is approximately 7.30 to 7.52; within this range, pH per se is not usually life-threatening. A pH outside this range is potentially life-threatening because of altered enzymatic activity and enhanced myocardial irritability, and direct steps should be taken to return the pH to normal (Source: http://www.mtsinai.org/pulmonary/books/physiology/chap7 1.htm). Thus, “imbalanced pH/imbalanced proton homeostasis” means in the context of this patent application is a local or systemic pH value other than 7.36 to 7.44.

“Similarity” is a further, more sophisticated measure of the relationship between two polypeptide sequences. In general, “similarity” means a comparison between the amino acids of two polypeptide chains, on a residue by residue basis, taking into account not only exact correspondences between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448, 1988, available as part of the Wisconsin Sequence Analysis Package).

Preferably, the BLOSUM62 amino acid substitution matrix (Henikoff S and Henikoff J G, Proc. Nat. Acad. Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5-25 in every 100 of the nucleotides of the in the reference sequence may be deleted, substituted or inserted, or any combination thereof, as hereinbefore described.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, Including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described.

The relationship between the number of nucleotide or amino acid differences and the Identity Index may be expressed in the following equation:
nₐ≦xₐ−(xₐ·I)
in which:
   nₐ is the number of nucleotide or amino acid differences,
   xₐ is the total number of nucleotides as listed in human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM 005282), mouse GPR4 (accession number: NM_{—}175668), human TDAG8 (accession number: NM_{—}003608) and mouse TDAG8 (accession number: NM_{—}008152) or amino acids in SEQ ID NO: 1, SEQ ID NO: 3, or SEQ ID NO: 4, respectively,
   I is the Identity Index,
   · is the symbol for the multiplication operator, and in which any non-integer product Of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotideor polypeptide that within the same species which is functionally similar.

“Fusion protein” refers to a protein encoded by two, unrelated, fused genes or fragments thereof.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### EXAMPLES

### Example 1

### Cloning of OGR1 and Stable Cell Line Generation

CCL39 hamster fibroblasts, HEK 293 human embryonic kidney cells, and MG63 human osteosarcoma cells (all cell lines from ATCC=American type:culture collection, Manassas, USA) are grown in a 1:1 mixture of bicarbonate-buffered DMEM and Ham's F12 medium supplemented with 10% fetal calf serum and antibiotics, in CO₂ atmosphere at pH 7.4. Primary cultures of human trabecular bone—derived preosteoblasts are established and cultured as described in detail before (Sottile V, et al., 2002, Bone, 30:699-704). Expression vectors for human OGR1 are prepared by cloning the cDNA of this receptor from human genomic DNA (U48405, NM_{—}003485) into pcDNA3.1 (+)/myc-His (Invitrogen, Basel, Switzerland). Site-directed mutagenesis is carried out using the Quick Change kit from Stratagene (Basel, Switzerland). For stable transfection, vectors are linearised with Pvul. Stable and transient transfections are carried out using the Effectene reagent (Qiagen, Basel, Switzerland). Stable cell populations expressing receptors are isolated following selection with antibiotic G418 (400 μg/ml). Expression of transgenes and membrane localisation is verified by performing immunocytochemistry using a FITC-labelled anti-myc antibody (Zymed/Stehelin & Cie, Basel, Switzerland).

### Example 2

### OGR1 is a Proton-Sensing G Protein Coupled Receptor Activating IP Formation

Inositol phosphate (IP) formation assay: Buffers and pH: Salt solutions for IP formation experiments are buffered either with HEPES alone (20 mM) or HEPES/EPPS/MES (8 mM each), to cover a wider pH range. HEPES is 4-(2-hydroxyethyl)piperazine-1-ethanesulfonic acid, EPPS is N′-(2-hydroxyethyl)piperazine-N′-3-propanesulfonic acid, MES is 2-(N-morpholino)ethanesulfonic acid. The pH of all solutions is adjusted to the indicated value at room temperature using a carefully calibrated pH meter (Metrohm, Herisau, Switzerland). All data in this report are referenced to pH at room temperature. To obtain pH at 37° C., 0.15 pH units should be subtracted for HEPES buffers in the range of pH 6.8-7.8 according to our calibration experiments. IP formation assay. Confluent cell cultures grown in 24 well plates are labelled with myo[³H]inositol (100 MBq/ml; ART/Anawa Trading, Wangen-Duebendorf, Switzerland) for 24 h in serum-free DMEM medium. Cells are then incubated at 37° C. in a buffered salt solution containing 130 mM NaCl, 0.9 mM NaH₂PO₄, 5.4 mM KCl, 0.8 mM MgSO₄, 1.8 mM CaCl₂, 25 mM glucose. Lithium (20 mM) is added to block inositol monophosphatase activity, leading to accumulation of IP₁ (Berridge M J, et al., 1982, Biochem J, 206:587-595; Berridge M J and Irvine R F, 1989, Nature, 341:197-205). Where indicated, bovine thrombin or SPC (both from Sigma, Buchs, Switzerland) is added 1 min prior to lithium addition. Unless otherwise stated, incubation is continued for 20 minutes. Cells are then extracted with ice-cold formic acid and total IPs separated from free inositol using batch column chromatography exactly as described before (Seuwen K, et al., 1988, EMBO J, 7:161-168).

Results: Following transient or stable expression of OGR1 a robust and apparently ligand-independent activation of phosphoinositide turnover in transfected cells is noticed. This effect persists in assay buffer devoid of either calcium, magnesium, phosphate, or sulfate, excluding that OGR1 is activated by any of these constituents. However, variations of buffer pH strongly affects inositol phosphate (IP) formation: CCL39 hamster fibroblasts stably expressing OGR1 are incubated in Hepes-buffered salt solution at different pH, and accumulation of IPs measured at 37° C. in the absence or presence of the inositol monophosphatase inhibitor lithium (Berridge M J, et al., 1982, Biochem J, 206:587-595; Berridge M J and Irvine R F, 1989, Nature 341:197-205). At pH 7.6, IP formation in the presence of lithium is close to background, however, lower pH resulted in a significant accumulation of IPs, which is linear over 30 minutes and maximal at pH 6.8. The pH of our standard assay buffer is 7.4, explaining why a significant basal activity has been observed in previous experiments. Activation of OGR1 by protons can be plotted as a standard concentration-response curve, covering a wider range of extracellular pH. Half-maximal activation of the receptor expressed in CCL39 hamster fibroblasts occurs at pH 7.48+/−0.04 (mean+/− sem; N=12). Activation appears highly co-operative, with a Hill coefficient of >2. Under acidic conditions (pH<6.5), IP formation declines again, reflecting the general pH-dependence of the phosphoinositide signalling system in CCL39 cells. SPC, the bioactive lipid reported to activate OGR1, does not stimulate the receptor in our hands, and does not affect pH-dependent stimulation of IP formation. The molecule is active, however, inhibiting forskolin-stimulated adenylyl cyclase in other cell systems, suggesting it acts on an as yet undefined receptor distinct from OGR1.

Importantly, untransfected cells or cells expressing other receptors do not show pH-dependent stimulation of phosphoinositide turnover. Similarly, the response to other receptor agonists is not positively modulated In the range of pH 7-8, as demonstrated here for thrombin, which activates endogenous receptors in CCL39 cells (Paris S and Pouyssegur J, 1986, EMBO J, 5:55-60). Pertussis toxin (PTX) does not inhibit IP formation measured in the presence of lithium at pH 7, strongly suggesting that OGR1 activates phosphoinositide turnover through Gq. The response to thrombin, which is additive to the signal elicited by OGR1 in these cells, is partially inhibited by PTX both at pH 7 and at pH 7.6, as expected from earlier work (Paris S and Pouyssegur J, 1986, EMBO J, 5:55-60).

Activation of OGR1 at neutral or slightly acidic pH is very strong, comparable to activation of other GPCRs by their cognate ligands. Strong pH-dependent activation of IP formation is also observed in transiently transfected HEK cells (half-maximal activation at pH 7.38+/−0.04 (mean+/−sem; N=4)) and in stably transfected HEK cells. Some pH-activated channels are strongly sensitive to temperature (Smith G D, et al., 2002, Nature, 418:186-190). However, IP formation rate for OGR1 measured at pH 7.6 and pH 7.0 is only minimally affected by temperature in the range of 35-42° C.

### Example 3

### Receptor Model of OGR1

Receptor model: Given the high similarity between OGR1 and other rhodopsin-like GPCRs the published rhodopsin structure (Palczewski K, et al., 2000, Science 289:739-745) as a template to build a homology model. The OGR1 primary sequence is aligned to bovine rhodopsin (bRHO), and for the indicated amino acid positions the side chains present in bRHO are substituted by the corresponding side chains of OGR1, using the SCWRL program (Bower M J, et al., 1997, J Mol Biol, 267:1268-1282). The resulting structure is refined applying molecular mechanics and dynamics, using the parm96.dat parameters of the AMBER force field (Cornell W D, et al., 1995, J Am Chem Soc, 117:5179-5197) with the distance-dependent dielectric function of epsilon=1*r, the minimax module of our own Wit!P software for energy refinements, and the sander_classic module of the AMBER6 package. During all computations, the Calpha atoms of the template are constrained in motion by fixing them with a harmonic potential or by constraining their position within 0.5 A (potential energy minimizations). The extracellular loops and all amino acid side chains are left free to move within the potential of the force field. A disulphide bond is introduced between residues CYS94 and CYS172. Intracellular loops are not included.

Results: Histidines should play an important role in the pH-dependent activation of OGR1. Indeed, inspection of a 3D model of the receptor indicates that several histidines might cluster at the extracellular surface, placed on top of helices 1, IV and VII, and in extracellular loops 1 and 2. All are conserved in the mouse, rat, and bovine sequence (see accession nos. XM_{—}138218, XM_{—}234483 and U88367, respectively). Specifically, the model predicts a direct hydrogen bond interaction between H20 and H269 in the unprotonated state, thus linking helix I and helix VII. A second interaction is possible between H17 and H84, linking the receptor N terminus to extracellular loop 1. However, given the flexibility of the extracellular loops, this interaction appears less likely to form than the Interaction between H20 and H269, and initial constraints are required in our model to make it fall in place. Additional histidines are found at other locations in the receptor, but their mutual interaction or strong electrostatic interactions with other residues seemed less obvious. We set out to mutate all potentially critical histidines individually to phenylalanine, and to measure pH-dependent receptor activation in transient transfection experiments. All receptor constructs are screened for expression of the recombinant protein by immunocytochemistry, using the C-terminal myc tag, and found to be expressed on the cell membrane at similar levels. Mutation of histidines 89, 159, 175 remains without major effect on receptor function. However, in agreement with our expectations, histidines 17, 20, 84, 269 are each required for normal sensitivity to pH change. In addition H169 turned out to be important. Mutation of these amino acids results in receptors that failed to stimulate IP formation at pH 6.8, however, upon exposure to more acidic pH activity can be restored. The proton concentration—response curves appears shifted to the right, i.e. sensitivity towards the ligand is reduced. In no case an increased pH-independent basal activity can be observed. A straightforward explanation for the involvement of H169 in proton sensing is difficult based on our model, given its position in extracellular loop 2, the geometry of which can not be well predicted at this stage. Mutation of H245 is not tolerated, leading to a severe, albeit not total, loss of function. According to our model this amino acid is located in helix VI facing the lipid environment, and may be required for overall structural integrity. In order to generate a receptor with a minimal number of histidines and still functional in the pH range of 7-7.8, we prepared and tested a H89, 159, 175F construct. The encoded protein indeed still functions as the wild type receptor in transiently transfected HEK cells, bottom right.

Pairs of histidines are able to co-ordinate Zn²⁺ and Cu²⁺ atoms, and this fact has been used to study GPCR structure-function (Elling C E, et al., 1995, Nature, 374:74-77). Based on our model we expect Zn²⁺ and Cu²⁺ to inhibit proton-dependent receptor activation, by stabilising the unprotonated state of the H20-H269 pair and possibly the H17-H84 pair. Indeed, micromolar concentrations of both ions strongly Inhibit OGR1-dependent IP formation stimulated at pH 6.9. In control CCL39 cells, Cu²⁺ remains without effect on thrombin-stimulated IP formation, Zn²⁺ ions led to a partial inhibition of this response.

### Example 4

### RT-PCR Expression Profiling of OGR1

RT-PCR expression profiling: Total RNA is prepared from cell cultures using the acid phenol method. RNA is DNAse-treated and reverse-transcribed using Superscript II (Life Technologies/Invitrogen, Basel, Switzerland). Parallel reactions for OGR1 and glyceraldehydes-3-phosphate dehydrogenase (GDPH) are set up with Expand High Fidelity Taq (Roche, Basel, Switzerland) using the following temperature cycling protocol: 30 sec denaturation at 94° C., 45 sec annealing at 65° C. (OGR1) or 55° C. (GPDH), 50 sec extension at 72° C.; 36 cycles for OGR1, 30 cycles for GPDH. GPDH is measured as internal standard for mRNA quantity. For OGR1, PCR reaction products are cloned and verified by sequencing. The following primers were used: OGR1 forward: 5′-CTGAGCCCATGAGGAGTGTG-3′, reverse: 5′-GGTAGGACGCCAGCAGCAGG-3′; GPDH forward: 5′-TTAGCACCCCTGGCCAAGG-3′, reverse: 5′-CTTACTCCTTGGAGGCCATG-3′.

Expression profiling by RT-PCR reveals the presence of mRNA for OGR1 in MG63 human osteosarcoma cells, and indeed we find that these cells respond strongly to neutral or acidic pH with IP formation. The signal is comparable in amplitude to that elicited by Bradykinin, which activates endogenous receptors in MG63 cells (Brechter A B, et al., 2002, Regul Pept, 103:39-51). Halfmaximal activation is observed at pH 7.46+/−0.01 (mean+/−sem; N=5). IP formation is insensitive to PTX pretreatment and is inhibited by micromolar concentrations of copper ions, which recapitulates the results described above for ectopic expression of OGR1 In fibroblasts. Similar results are obtained for primary human osteoblast precursors isolated from trabecular bone. Half-maximal activation in primary cells occurs at pH 7.41+/−0.02 (mean+/−sem; N=3).

### Example 5

### Expression Studies of OGR1

Recombinant receptors are detected using a FITC-labelled anti-myc antibody (No. 132511, Zymed/Stehelin & Cie, Basel, Switzerland). To detect the plasma membrane marker annexin V, an alexa-labelled antibody (No. A13202, Juro Supply, Luzern, Switzerland) is used. Nuclei are stained with the dye H33258 (Sigma, Buchs, Switzerland).

Experiments on bone tissue are performed on 4 μm sections of paraffin-embedded organs collected from 6 month old female Wistar rats. Sections are deparaffinized in xylol and antigens unmasked using pepsin digestion (10 min, Sigma, Buchs, Switzerland).

Endogenous peroxidase is blocked by a 5 minute Incubation with 3% hydrogen peroxide followed by 10% goat serum for 1 hour. Immunohistochemical detection is performed using a rabbit polyclonal antibody (1:100, 3 h incubation) developed by Lifespan Biosciences Inc. (Seattle, USA) directed against the peptide epitope CFVSETTHRDLARLRG (SEQ ID NO: 2), which is identical on human and rat OGR1. Staining is revealed using the ABC peroxidase staining Kit (Santa Cruz Biotechnology/LabForce, Nunningen, Switzerland).

Immunohistochemistry on rat bone sections (as described in the above paragraph) located OGR1 in osteoblasts and osteocytes.

### Example 6

### OGR-1 Knock-Out Mice

Generation of OGR-1 Knock-Out Mice:

Generation of a targeting vector for homologous recombination: The mouse OGR-1 transcript mCT51440 was identified in the mouse genome Celera database to correspond to a locus on mouse chromosome 12 designated as mCG51257. Primers are designed according to the Celera sequence information to amplify genomic DNA used for the generation of a targeting vector for homologous recombination and knock-out of the OGR-1 gene. Sequences of primers and conditions for the amplification of two flanking genomic regions are:

For region 1: forward primer: TS145: CTATCTGCATGTGGAGCCCC and reverse primer: TS140: CTGGCAGGATAGGTCACCAT. PCR is performed using the KOD Hot Start DNA polymerase (Novagen/Juro, Luzeme, Switzerland) In a T3 PCR Biometra thermocycler. In short, 200 ng 129Sv genomic DNA are prepared in a total volume of 50 μl together with 200 μM dNTP mix, 600 nM forward primer, 600 nM reverse primer, 5 μl 10×PCR buffer (1 mM MgSO₄) and 1 μl KOD Hot Start DNA polymerase. Settings for the amplification of genomic DNA were 94° C. for 3 min., followed by 35 cycles of 94° C. for 30 sec., 58° C. for 30 sec., 68° C. for 5 min, followed by a final extension at 68° C. for 5 min. Finally, the reaction is cooled down to 4° C. 4 ul of the PCR product are subcloned using the Zero blunt Topo PCR cloning kit (Life Technologies/Invitrogen, Basel, Switzerland) according to the manufacturers instructions resulting in pTOPO-region1 which is confirmed by sequencing.

For region 2: forward primer: TS143: GCTTGCATGGTGGCTGTCTC and reverse primer: TS142: TACAACACCACCTGCACAGA. PCR is performed in a Perkin Elmer PE9600 PCR thermocycler with Pfu DNA polymerase (Promega, Wallisellen, Switzerland). Briefly, 200 ng of 129Sv genomic DNA are prepared in 50 ul together with 200 uM dNTP, 1 uM forward primer and 1 uM reverse primer, 1×Pfu DNA polymerase buffer (containing 2 mM MgSO₄), 5% Dimethylsulfoxide (DMSO) and 1.25 u Pfu DNA polymerase. Settings for the touch down amplification of the genomic DNA are 94° C. for 3 min., 10 cycles of 94° C. for 30 sec., 68° C. (−1° C./cycle) for 30 sec., 68° C. for 5 min, followed by 25 cycles of 94° C. for 30 sec., 55° C. for 30 sec., 68° C. for 5 min and a final step at 68° C. for 10 min. Finally, the reaction is cooled down to 4° C. 1 ul of this PCR reaction is amplified by nested PCR using forward primer TS167: CCATCGATGCTTGCCTCTAAACTAGTCT and reverse primer TS168: ATAGTTTAGCGGCCGCCTCTACTGTCCTTGTGGCTT with Pfu polymerase under the same conditions as above. 4 ul of the PCR product are subcloned using the Zero blunt Topo PCR cloning kit (Invitrogen, Basel, Switzerland) according to the manufacturers constructions and confirmed by sequencing. For the generation of the targeting vector for homologous recombination a genomic fragment 1 is amplified using region 1 as template and primers forward: TS170: CCCAAGCTTAGAGCAGGTGACTGTGCATA and reverse: TS171: CCGCTCGAGCTTTGGGCCAGAAGGAGCCT. 10 ng of pTopo-region 1 are used as template in the PCR mix which is as described for the amplification of region 1 using KOD Hot Start polymerase. PCR is performed in a T3 PCR Biometra thermocycler and settings are: 94° C. for 2 min., followed by 31 cycles of 94° C. for 15 sec., 58° C. for 30 sec., 74° C. for 1 min 30 sec, followed by a final extension at 74° C. for 1 min. The amplified PCR fragment is purified using the PCR purification kit (Qiagen, Basle, Switzerland) according to the manufacturers instructions, digested with the restriction enzymes HindIII and XhoI, ligated into the HindIII/XhoI digested vector pRAY-2 (Accession number U63120), and confirmed by sequencing.

A second genomic fragment is amplified using 1 μl pTopo-region 2 as template and forward primer TS167 as well as reverse primer TS168. PCR mix is as described above for the amplification of region 1 and settings are: 94° C. for 2 min., followed by 35 cycles of 94° C. for 15 sec., 55° C. for 30 sec., 68° C. for 1 min 30 sec, followed by a final extension step 68° C. for 5 min. The amplified PCR fragment is subcloned using the Zero blunt Topo PCR cloning kit and confirmed by sequencing. After digestion using the restriction enzymes ClaI and NotI, the PCR fragment is ligated Into the ClaI/NotI digested vector pRAY-2 containing fragment 1. The resulting OGR-1 targeting vector is confirmed by sequencing.

ES cell culture and transfection: The final OGR-1 targeting vector is linearized using the restriction enzyme ScaI and 17 μg are electroporated into 1.5×10e7 129S3 mouse embryonic stem cells (ES cells) at 250 V and 500° F. The cells are cultured in 6 cm dishes containing primary inactivated embryonic fibroblast cells; Selection medium containing G418 (200 μg/ml, Gibco/Invitrogen, Basel, Switzerland) is added 24 h after electroporation. Resistant ES cell colonies are isolated 10 days after electroporation and analyzed by nested PCR to identify homologous recombination events of the targeting construct into the OGR-1 locus (genotyping by PCR).

Genotyping by PCR: ES cells are extracted in 50 μl lysis buffer (0.05% SDS, 50 μg/ml proteinase K, 10 mM Tris/HCL, pH7.4) and diagnostic PCR is performed using 1 μl crude ES cell extract in a total volume of 25 μl together with 200 μM dNTP mix, 600 nM forward primer, 600 nM reverse primer, 1×Taq PCR master mix (Qiagen, Basel, Switzerland), in a Tgradient PCR Biometra thermocycler. Primers used for PCR are forward: TS207: TGATATTGCTGAAGAGCTTGGCGGC and reverse: TS203: CCAGGGTAGCTTTGCAACATGC for the first amplification as well as forward: TS208: AGCGCATCGCCTTCTATCGCC and reverse: TS204: ATGGGCTTTGCCATGAGGCAG for the nested reaction. PCR conditions are 95° C. for 3 min; 35 cycles of 95° C. for 30 sec; 62° C. for 45 sec; 72° C. for 2 min. For the nested reaction 1 μl of the first reaction are amplified at 95° C. for 3 min; 25 cycles of 95° C. for 30 sec; 62° C. for 45 sec; 72° C. for 2 min. Finally, 10 μl of the nested PCR reaction are analysed on a 1% agarose gel.

Southern genotyping of ES cells: In order to generate a probe suitable for Southern hybridization, a OGR-1 genomic region is amplified by PCR using 100 ng pTopo-region 1 as template. Primers used for amplification are forward: TS146: CAAGGGCAGGGGAGTCAAGG and reverse: TS159: TAATTATTCTACTTTATTAC. The PCR mix was as described above using Pfu DNA polymerase. Settings for PCR were 94° C. for 2 min., 10 cycles of 94° C. for 15 sec., 55 (−1° C./cycle) for 15 sec., 72° C. for 30 sec, followed by 25 cycles of 94° C. for 15 sec., 45° C. for 15 sec., 72° C. for 30 sec. Finally, the reaction was cooled down to 4° C. The amplified PCR fragment was purified as described above using the PCR purification kit.

Genomic DNA from 129S3 ES cells is digested over night using 12 μg genomic DNA and the restriction enzymes SspI/RsrlI or SspI/XhoI. The digested DNA is separated on a 0.9% agarose gel and blotted to a Hybond N+ membrane (Amersham, Dübendorf, Switzerland). Random prime labelling of the DNA probe with ³²P-dCTP is performed using the Amersham rediprime II kit as described by the manufacturer. Membranes are hybridized over night at 65° C. in PerfectHyb Plus hybridization buffer (Sigma, Buchs, Switzerland), washed in 0.5×SSC; 0.1% SDS and imaged by exposure to a Kodak X—O-Mat film.

A neo probe corresponding to the NheI/BamHI fragment of pRAY2 vector is used under the same conditions to confirm the unique integration of the targeting construct.

Karyotype analysis: ES cells are splitted the day before chromosome spreads are performed. For chromosome spreading, ES cells are treated with 10 μg/ml Colchicin (KaryoMax, Gibco/Invitrogen, Basel, Switzerland) for 4.5 h at 37° C. and 10% CO₂ followed by an incubation for 10 min at room temperature in prewarmed (37° C.) 0.56% KCl. Fixation is performed by using ice cold methanol/acetic acid (3:1). Spreading of the chromosomes is analyzed on a microscope.

Blastocyste injection and breeding: Targeted ES cells are injected into C57BI/6 host blastocysts and transferred into pseudopregnant C57BI/6×Balb/c N1 foster mothers. Chimeric offspring are identified by coat pigmentation. Male chimeras are mated with 129S3 wildtype females. The offspring are tested for germline transmission by genotyping PCR.

### Example 7

### Screening Assay for Agonists or Antagonists of OGR1

Method: Cytoplasmic calcium transients are recorded using the calcium indicator Fluo-3 and a fluorescence-imaging plate reader (Molecular Devices/BucherBiotech, Basel, Switzerland).

CCL39 cells transfected with OGR1 are loaded with the acetoxymethylester of the dye (2 μg/ml) for 1 h at 37° C. in full medium containing 5 mM probenicide to inhibit the multidrug resistance transporter. Cells are then washed and maintained in buffered salt solution containing 130 mM NaCl, 0.9 mM NaH₂PO₄, 5.4 mM KCl, 0.8 mM MgSO₄, 1.8 mM CaCl₂, 25 mM glucose, 5 mM HEPES at pH 7.6 for 45 min at room temperature. To identify receptor antagonists, test compounds are added during the last 15 minutes of this incubation. After transfer to the reader a baseline is recorded (Fb), and cells are then stimulated by addition of an appropriate amount of stronger buffer (20 mM HEPES, pH 6), inducing acidification.

Alternatively, test compounds are added at this stage to identify agonistic molecules. The fluorescence readout F is normalised calculating F′=(F−Fb)/Fb.

Result: Shift of extracellular pH from pH 7.6 to more acidic values results in rapid and transient elevations of intracellular calcium concentration, indicating the release of the cation from intracellular stores. The amplitude of the response is pH—dependent, Increasing with acidity. Half-maximal activation occurs at pH 7.20+/−0.04 (mean+/−sem; N=3). Receptor antagonists block the fluorescence signal.

### Example 8

### Cloning of GPR4 and Stable Cell Line Generation

Human GPR4 is amplified from genomic DNA using primers GPR4F and GPR4R (5′ CACC ATG GGC AAC CAC ACG TGG GAG GGC TGC 3′ and 5′ TCA TTG TGC TGG CGG CAG CAT CTT CAG CTG CA 3′ respectively). The PCR reaction mixture contains 0.2 mM dNTPs, 1×PCR buffer containing 1.5 mM MgCl₂, 0.5 Units Taq DNA polymerase, 40 pmol each primer and sterile water in a total volume of 50 μl. The template for this reaction is human genomic DNA (Promega, Southampton, UK). PCR is performed using a Biometra T3 Thermocycler using the following cycling conditions: Denaturation at 95° C. for 2 min followed by 35 cycles of denaturation at 95° C. for 15s, annealing at 60° C. for 15 s, and extension at 72° C. for 1 min. A final extension at 72° C. is performed for 7 min. The 1.1 kb PCR product is purified and cloned pcDNA3.1 DN5-His-TOPO (Invitrogen, Paisley, UK).

Stable cell lines expressing GPR4 are generated in either CHOK1 CRE-luc cells or CCL39 CRE-luc cells which stably express a cAMP-dependent luciferase reporter to detect changes in cAMP in response to ligand. Stable cell lines are generated in the above cell lines by transfecting the GPR4 cDNA expression construct using Lipofectamine 2000 (Invitrogen, Paisley, UK) according to the manufacturers protocol. Transfected cells are selected in the presence of 400 μg/ml or 1 mg/ml of G418. After 3 weeks of antibiotic selection, individual clones are picked and expanded for further analysis.

### Example 9

### GPR4 is a Proton-Sensing G Protein Coupled Receptor Activating cAMP Formation

cAMP formation assay: Confluent cell cultures grown in 24 well plates are labelled with [³H]adenine (100 MBq/ml; Amersham, Dübendorf, Switzerland) for 4 h in serum-free DMEM medium. Cells are then Incubated at 37° C. in buffered salt solution as described above for the IP assays (Example 2). Where Indicated, the phosphodiesterase inhibitor isobutylmethylxanthine (IBMX, 1 mM) is added to allow accumulation of cAMP. Incubation time is 15 minutes. Cells are then extracted with ice-cold trichloroacetic acid and cAMP separated from free adenine and ATP using batch column chromatography (Salmon Y, 1979, Adv. Cycl. Nucleot Res, 10: 35-55.

Result: Expression studies and cAMP formation assays as described above, show that this receptor indeed responds to pH shifts in a very similar range as OGR1. SPC, which is reported to be a ligand for GPR4 (Zhu K, et al., 2001, J Biol Chem, 276:41325-41335), does not modulate pH-dependent stimulation and does not activate cAMP formation on its own. Half-maximal activation of cAMP formation by GPR4 in transiently transfected HEK293 cells occurs at pH 7.55+/−0.02 (mean+/−sem; N=6).

### Example 10

### Screening Assay for Agonists or Antagonists of GPR4

Cells co-expressing GPR4 and a cAMP luciferase reporter, as described in Example 8, are plated at 10-20,000 cells per well in white 96 well plates the day before performing the assay and incubated overnight at 37° C. in the presence of 5% CO₂. Cells are washed once with 200 μl HBS buffer (130 mM NaCl, 0.9 mM NaH₂PO₄, 0.8 mM MgSO₄, 1.8 mM CaCl₂, 5.4 mM KCl, 25 mM glucose, 20 mM HEPES) at the appropriate pH containing the to be tested compound as appropriate, e.g. any in-house or commercially available compound library may be used. Plates are incubated 4-5 h at 37° C. in a non-CO₂ incubator. After 4-5 h buffer is aspirated from the cells and cells are washed once with 200 μl of HBS pH 7.4. Cells are lysed by adding 100 μl of HBS pH 7.4 and 100 μl of Steady Glo luciferase reagent (Promega, Southampton, UK) and placed on rotating platform for 25-30 min with vigorous rotation. Luminescence is measured using a luminometer. In acidic pH buffer (pH 6.8 or pH 7.0) a high luminescence signal is produced. Antagonists are characterised in the assay by a decrease in luminescence signal compared to the no compound control, e.g. in acidic pH buffer (pH 6.8 or pH 7.0).

### Example 11

### RT-PCR Expression Profiling of GPR4

RNA extraction and first strand cDNA synthesis: RNA is prepared from cells using RNeasy extraction kits (Qiagen, Crawley, UK) according to the manufacturers protocol. Cells used are primary human bronchial epithelial cells (HBECs), differentiated HBECs, primary human lung fibroblasts, primary human bronchial smooth muscle cells (BSMC), human peripheral blood T-cells and human peripheral blood neutrophils. Primary human cells are either purchased from Biowhittaker or, where indicated, isolated from peripheral human blood according to standard procedures. First strand cDNA is prepared from total RNA isolated from cells using the reagents and protocol provided in the first strand cDNA synthesis kit (Roche, Lewes, UK).

RT-PCR: The GPR4 receptor is profiled in cDNA derived from tissues and in different cell types described above by reverse transcriptase polymerase chain reaction (RT-PCR). Tissue cDNAs used for RT-PCR profiling are purchased from Clontech (Basingstoke, UK). Each reaction mixture contains 0.2 mM dNTPs, 1×PCR buffer containing 1.5 mM MgCl₂, 0.5 Units Taq DNA polymerase, 50 pmol each primer and deionised water in a total volume of 25 μl. Template cDNA used is either from commercial cDNA derived from tissue samples from Clontech panel I and II (2.5 μl) or from cDNAs prepared from cell types as described in the previous section (1 μl). The GPR4 RT-PCR primers used are as follows: 5′ TGG GCG TCT ACC TGA TGA A 3′ and 5′ GGG TTT GGC TGT GCT GTT 3′. Cycling is carried out in 0.2 ml tubes using a Biometra Trio PCR machine. Cycling conditions are as follows: Denaturation at 94° C. for 1 min 45s for 1 cycle then 35 cycles of denaturation at 94° C. for 15s, annealing at 60° C. for 15s, extension at 72° C. for 30s. Reactions are analysed on a 1.5% agarose gel and stained with ethidium bromide. Control RT-PCR reactions are performed with primers specific to the housekeeping gene GAPDH.

Quantitative RT-PCR: Messenger RNA levels in total RNA samples are measured by TaqMan quantitative RT-PCR. Primers and probes are obtained as pre-optimized reagents purchased from Applied Biosystems (Warrington, UK). Quantitative RT-PCR reactions are performed in triplicate in 25 μl final volumes and contained 1×TaqMan Universal PCR master mix target cDNA preparation in each reaction. Experiments are performed using an ABI PRISM 7700 sequence detector (Applied Biosystems, Warrington, UK) and analysed using ABI PRISM 7700 Sequence Detection System software. Amplification conditions are as follows: 50° C. for 2 min and 95° C. for 10 min followed by 40 cycles of 95° C. for 15 s and 60° C. for 1 min. The data are quantified by extrapolation from the standard curve, generated using a cDNA pool of 12 human tissues, normalised to the housekeeping gene glyceraldehyde-3-phosphate dehydrogenase (GAPDH).

TABLE 1
Summary of expression profiling data of GPR4

| Tissue or Cell Type | GPR4 expression |
|---|---|
| Brain | − |
| Heart | + |
| Kidney | +++ |
| Liver | +++ |
| Lung | +++ |
| Pancreas | +++ |
| Placenta | − |
| Skeletal Muscle | + |
| Colon | ++ |
| Ovary | − |
| Leukocyte | − |
| Prostate | ++ |
| Small Intestine | +++ |
| Spleen | +++ |
| Testis | ++ |
| Thymus | ++ |
| Monocytes | ++ |
| Bronchial epithelial cells | + |
| Differentiated bronchial epithelial cells | + |
| Lung fibroblasts | + |
| Airway smooth muscle cells | ++ |
| Neutrophils | +++ |
| T-cells | + |

### Example 12

### Immunolocalisation Studies of GPR4

Recombinant receptors are detected using a FITC-labelled anti-myc antibody (No. 132511. Zymed/Stehelin & Cie, Basel, Switzerland). To detect the plasma membrane marker annexin V, an alexa-labelled antibody (No. A13202, Juro Supply, Luzern, Switzerland) is used. Nuclei are stained with the dye H33258 (Sigma, Buchs, Switzerland).

Experiments on human tissue are performed on 3 μm paraffin sections of lung tissue mounted on polysine slides. Paraffin sections are first treated with xylene and industrial methylated spirit (IMS) to 70% IMS and endogenous peroxidase is blocked with 0.05% hydrogen peroxide in methanol. Sections are stained after microwave irradiation In Dako pH6 antigen retrieval solution. Unwanted background staining is reduced with Dako serum-free protein blocker for 5 min. Immunohistochemical detection is performed using rabbit polyclonal antibodies (1:500, overnight at 4° C.) developed by Lifespan Biosciences Inc. (Seattle, USA) directed against either the peptide epitope RSDVAKALHNLLRFLASDK (SEQ ID NO: 5), which is Identifies human GPR4, or alternatively using a rabbit polyclonal antibody developed directed against the peptide epitope DELFRDRYNHTFCFEKFPME (SEQ ID NO: 6), which is identifies human and mouse GPR4. Staining is revealed by first treatment with Dako biotinylated swine anti-rabbit immunoglobulins (1:200) in Dako diluent for 30 min. After a wash step, sections are treated with Dako streptavidin-biotin peroxidase complex (SABCpx). Sites of reaction are visualised with diaminobenzidine (DAB). Nuclei are counterstained with Cole's haematoxylin.

TABLE 2
Protein Expression of GPR4 by immunohistochemistry

| | Location of | GPR4 |
|---|---|---|
| Tissue | expression | expression |
| Lung, asthma | Respiratory | ++ |
| | epithelium | |
| | Neutrophils | +++ |
| Lung, normal | Respiratory | −/+ |
| | epithelium | |
| | Neutrophils | +++ |
| | Alveolar | + |
| | macrophages | |

The data above suggests that expression of GPR4 protein is up-regulated In the respiratory epithelium of asthmatics suggesting there may be an association with the disease. GPR4 protein is either absent or only weakly expressed in lung tissue of normal subjects. Expression of GPR4 protein in alveolar macrophages, neutrophils and bronchial smooth muscle in normal lung tissue confirms the results at the mRNA level (Table 1).

### Example 13

### Functional Assay for GPR4 (Neutrophil Chemotaxis Assay)

Assays are performed in a 96-well plate format according to previously published method (Frevert C W, et al., J Immunology Methods, 1998, 213:41). All cell buffers are obtained from Invitrogen (Paisley, UK). Calcein-AM dye is obtained from Molecular Probes (Invitrogen, Paisley, UK). Neutrophils are isolated as previously described (Haslett C, et al., 1985, Am J Path, 119:101). Briefly, citrated whole blood is mixed with 4% dextran-T500 and allowed to stand on ice for 30 min to remove erythrocytes. Granulocytes (PMN) are separated from peripheral blood mononuclear cells by Ficoll-Paque density gradient centrifugation. Any erythrocyte contamination of PMN pellet is removed by hypotonic shock lysis. Isolated granulocytes (neutrophils) are labelled with the fluorochrome calcein-AM (5 μg). The labelled neutrophils are then mixed with test compounds (0.001-1000 nM) diluted in DMSO and incubated for 10 min at room temperature. The agonist (buffer at pH 6.8 or pH 7.0) is placed in the bottom chamber of a 96-well chemotaxis chamber. The polycarbonate filter (5 μm) is overlaid on the plate and the cells and antagonist, if used, is loaded on the top filter. The cells are allowed to migrate for 90 min at 37° C. in a humidified incubator with 5% CO₂. At the end of the incubation period, migrated cells are quantified using a multi-well fluorescent plate reader (Fluoroskan II, Labsystems) at 485 nm excitation and 538 nm emission. Positive control cells (cells without added antagonist) represent the maximum chemotactic response of the cells. While the negative control (unstimulated) no agonist, i.e pH>=7.4 is added to the bottom chamber. The difference between the positive control and negative control represents the chemotactic activity of the cells. Anatgonists are characterised in the assay by a decrease In the chemotactic response to acidic pH compared to the no compound control.

### Example 14

### Functional Assay for GPR4 (Shape Change Assay)

Agonist (pH dose response) curves are determined as follows: Aliquots of granulocytes (80 μl of 4-5×10⁵ cells) are mixed with 10 μl of assay buffer (1×PBS, pH 7.4, plus 0.1% BSA) in 1.2 ml cluster tubes. Samples are shaken gently and incubated for 5 min at 37° C. (in a water bath). After which time agonist is added to all tubes except the zero control to which 10 μl assay buffer is added. Samples are shaken gently and incubated for a further 5 min at 37° C. in water bath. 250 μl of ice cold, optimised, 0.25%, CellFix™ solution (Becton Dickinson, Oxford, UK) is then added to each tube to terminate the reaction and maintain the cell shape change until analysis. Tubes are shaken gently and incubated on ice for 10 min before data is acquired on a FACSCalibur flow cytometer. FSC/SSC plots are acquired first, followed by FSC/FL-2 plots using the gated granulocytes from the first plot. Neutrophils are distinguished from eosinophils on the FL-2 plot, as the latter have a higher autofluorescence.

For antagonist (No compounds yet) assays, aliquots of granulocytes (80 μl of 4-5×10⁵ cells) are mixed with 10 μl of 10× concentrated compound (0.1-1000 μM final) containing constant concentration of DMSO (max 10%) in 1.2 ml cluster tubes, samples are shaken gently and incubated for 5 min at 37° C. (in a water bath). Agonist (buffer at pH 6.8 or 7.0) is added to all tubes except the zero compound/zero agonist control, to which 10 μl assay buffer is added.

Samples are shaken gently in the rack and were incubated for a further 5 min in the 37° C. water bath. 250 μl of ice cold, optimised, 0.25%, CellFix™ solution is then added to each tube and the rack is shaken gently and incubated on ice for 10 min before data is acquired on the flow cytometer. Neutrophils are distinguished from eosinophils on the FL-2 plot, as the latter have a higher autofluorescence. Antagonists are characterised in the assay by a decrease in observed shape change compared to the no compound (I.e. pH 6.8 or pH 7.0 buffer only) control.

### Example 15

### Gene Expression Profiling Screen for GPR4

Potential therapeutic targets for angiogenesis are identified by selecting genes that show correlated expression with recognized marker genes for endothelial cells. This marker gene set comprises, but is not limited to, CDH5, VWF, KDR, TIE, TEK, PTPRB, ANGPT2 (approved gene symbols according to the Human Genome Organisation/Gene Nomenclature Committee (HUGO/HGNC)).

Gene expression data are obtained by DNA microarray analysis using Affymetrix (3380 Central Expressway, Santa Clara, Calif. 95051, USA; http://www.affymetrix.com/index.affx) oligonucleotide array technology.

Data are imported to the software tool GeneSpring (Silicon Genetics, 2601 Spring Street, Redwood City, Calif. 94063, USA; http://www.silicongenetics.com/cgi/SiG.cgi/index.smf).

For each of the marker genes other genes are identified that show correlated expression by using the Pearson Correlation method, a statistical measurement of association. Correlation coefficients of 0.7 to 1 are considered to indicate strong correlation.

Among genes considered as potential drug targets, the analysis reveales 12 GPCRs with a correlation coefficient>0.7.

| | | Marker | | |
|---|---|---|---|---|
| Approved | Alias | Gene with | | |
| Gene Symbol | Gene | highest | Corre- | |
| (HUGO/HGNC) | Symbols | correlation | lation | Ligand |
| GPR116 | | VWF | 91% | Orphan |
| ELTD1 | ETL | TIE | 89% | Orphan |
| CALCRL | | CDH5 | 88% | Adrenomedullin |
| FZD4 | | VWF | 82% | Wnt |
| GPR143 | OA1 | ANGPT2 | 79% | Orphan |
| GPR126 | VIGR | CDH5 | 73% | Orphan |
| GPR4 | | VWF | 73% | Protons |
| EDG1 | | VWF | 72% | S1P |
| F2R | PAR1 | ANGPT2 | 72% | Thrombin |
| CMKOR1 | RDC1 | CDH5 | 72% | Adrenomedullin |
| GPR124 | TEM5 | TEK | 70% | Orphan |
| GPR56 | TM7LN4 | VWF | 70% | Orphan |

### Example 16

### Endothelial cell Proliferation Assay for GPR4

Primary human dermal microvascular endothelial cells (HDMECS) and primary human umbilical vein endothelial cells (HUVECS) are purchased from PromoCell, Heidelberg, Germany.

HDMECs are grown In Endothelial Cell Basal Medium MV supplemented with SupplementPack/Endothelial Cell Growth Medium MV and 5% fetal calf serum (FCS), HUVECs are grown in Endothelial Cell Basal Medium supplemented with SupplementPack/Endothelial Cell Growth Medium plus 12.5% FCS.

Endothelial Cell Basal medium is used as minimal or starvation medium.

Mouse primary cells are isolated from mouse lungs as described by Reynolds LE et al, Nat. Med. 2002 January;8(1):27-34. Briefly, mouse lungs are minced, collagenase-digested (Gibco), strained and the resulting cell suspension plated on flasks coated with a mixture of 0.1% gelatin (Sigma), 10 mg/ml fibronectin (Sigma) and 30 μg/ml Vitrogen (Collaborative Biomedical Research). Endothelial cells are purified by a single negative (FCγ-RII/III antibody; Pharmingen) and two positive (ICAM-2; Pharmingen) cell sorts using anti-rat IgG-conjugated magnetic beads (Dynal, Wiltshire, UK) producing a >97% pure population.

Proliferation is measured as a function of extracellular pH in endothelial cells stimulated with different growth factors such as VEGF₁₆₅ (10 ng/ml) and bFGF (0.5 ng/ml). In addition, similar experiments are carried out with endothelial cells transfected with an siRNA against GPR4 or in the presence of compounds targeting GPR4 in order to elucidate the specific role of GPR4 in this process. Proliferation of endothelial cells is used as an in vitro readout for angiogenesis. Inhibition of proliferation in endothelial cells represents one way of reducing angiogenesis.

An endothelial cell proliferation assay, based on BrdU incorporation is used (Biotrak Cell Proliferation Elisa System V.2, Amersham, England). Subconfluent HUVEC are seeded at a density of 5×10³ cells per well into 96-well plates coated with 1.5% gelatin and then incubated at 37° C. in growth medium. After 24 h, the medium in the wells which are to be incubated with growth factors, is replaced by basal medium containing 1.5% FCS, the medium in the remaining wells is replaced by growth medium. After another 24 h, the medium is replaced with fresh medium containing the same amounts of FCS (5% or 1.5%) as before plus minus the compound to be tested and the specific growth factor. As a baseline control for the effects of the growth factors, wells without growth factor are also included. After 24 h of incubation, BrdU labeling solution is added and cells incubated for further 24 h before fixation, blocking and addition of peroxidase-labeled anti-BrdU antibody. Bound antibody is detected using 3,3′5,5′-tetramethylbenzidine substrate, which forms a coloured reaction product that is quantified spectrophotometrically at 450 nm. Every sample is run in triplicate.

Cells transfected with siRNA are used 24 h after transfection.

### Example 17

### Endothelial Cell Apoptosis Assay for GPR4

Primary human dermal microvascular endothelial cells (HDMECs) and primary human umbilical vein endothelial cells (HUVECs) are purchased from PromoCell, Heidelberg, Germany.

HDMECs are grown in Endothelial Cell Basal Medium MV supplemented with SupplementPack/Endothelial Cell Growth Medium MV and 5% fetal calf serum (FCS), HUVECs are grown in Endothelial Cell Basal Medium supplemented with SupplementPack/Endothelial Cell Growth Medium plus 12.5% FCS.

Endothelial Cell Basal medium is used as minimal or starvation medium.

Mouse primary cells are isolated from mouse lungs as described by Reynolds L E et al, Nat. Med. 2002 January;8(1):27-34. Briefly, mouse lungs are minced, collagenase-digested (Gibco), strained and the resulting cell suspension plated on flasks coated with a mixture of 0.1% gelatin (Sigma), 10 mg/ml fibronectin (Sigma) and 30 μg/ml Vitrogen (Collaborative Biomedical Research). Endothelial cells are purified by a single negative (FCγ-RII/Ill antibody; Pharmingen) and two positive (ICAM-2; Pharmingen) cell sorts using anti-rat IgG-conjugated magnetic beads (Dynal, Wiltshire, UK) producing a >97% pure population.

Apoptosis is measured in endothelial cells as a function of extracellular pH. In addition, similar experiments are carried out with endothelial cells transfected with an siRNA against GPR4 or in the presence of compounds targeting GPR4 in order to elucidate the specific role of GPR4 in this process. Serum starvation is used a control for inducing apoptosis and VEGF₁₆₅ (10 ng/ml) is used as a control to rescue cells from apoptosis. Induction of apoptosis in endothelial cells represents one way of inhibiting angiogenesis.

Apoptosis is measured using the Cell Death Detection ELISA^{PLUS} system (Roche Diagnostics,) as per manufacturer's protocol. Briefly, after serum starvation, treatment with pH shift, growth factors, compounds or GPR4-siRNA the cells (HUVECs or HDMVECs) are gently lysed to release only the cytoplasmic contents with 100 μl/well of lysis buffer at 37° C. for 30 min and spun at 200 g for 10 min. 20 μl of the supernatant is transferred to the ELISA plate and 80 μl of the Immunoreagent containing the antibodies is added and incubated with shaking (300 rpm) for 2 hr in a Titer Plate shaker. The cells are washed 3 times with incubation buffer provided in the kit. 100 μl of ABTS color reagent is added and OD is measured at 405 nm after 10 min using a Spectromax 190 Microplate Spectrophotometer and ABTS solution as a blank. Every sample is run in triplicate.

### Example 18

### Endothelial Cell Migration Assay for GPR4

Primary human dermal microvascular endothelial cells (HDMECs) and primary human umbilical vein endothelial cells (HUVECs) are purchased from PromoCell, Heidelberg, Germany.

HDMECs are grown in Endothelial Cell Basal Medium MV supplemented with SupplementPack/Endothelial Cell Growth Medium MV and 5% fetal calf serum (FCS), HUVECs are grown in Endothelial Cell Basal Medium supplemented with

SupplementPack/Endothelial Cell Growth Medium plus 12.5% FCS.

Endothelial Cell Basal medium is used as minimal or starvation medium.

MS1 is a mouse pancreatic islet endothelial cell line established transduced with the polyomavirus middle T antigen and is purchased from ATCC. MS1 cells are grown in Dulbecco's modified Eagle's medium with 4 mM L-glutamine, 1.5 g/L sodium bicarbonate and 4.5 g/L glucose plus 5% FCS.

bEnd3 is a mouse brain endothelial cell line established transduced with the SV40 large T antigen and is purchased from ATCC. bEnd3 cells are grown in Dulbecco's modified Eagle's medium with 4 mM L-glutamine, 1.5 g/L sodium bicarbonate and 4.5 g/L glucose plus 10% FCS.

Mouse primary cells are isolated from mouse lungs as described by Reynolds LE et al, Nat. Med. 2002 January;8(1):27-34. Briefly, mouse lungs are minced, collagenase-digested (Gibco), strained and the resulting cell suspension plated on flasks coated with a mixture of 0.1% gelatin (Sigma), 10 mg/ml fibronectin (Sigma) and 30%g/ml Vitrogen (Collaborative Biomedical Research). Endothelial cells are purified by a single negative (FCγ-RII/III antibody; Pharmingen) and two positive (ICAM-2; Pharmingen) cell sorts using anti-rat IgG-conjugated magnetic beads (Dynal, Wiltshire, UK) producing a >97% pure population.

Migration of endothelial cells towards a specific growth factor such as VEGF (10 ng/ml) or S1P (100 nM) is measured as a fuction of extracellular pH. In addition, similar experiments are carried out with endothelial cells transfected with an siRNA against GPR4 or in the presence of compounds targeting GPR4 in order to elucidate the specific role of GPR4 in this process. Migration of endothelial cells is used as an in vitro readout for angiogenesis. Inhibition of migration of endothelial cells represents one way of inhibiting angiogenesis.

Migration assays are performed using the BD Falcon HTS FluoroBlok Multiwell Insert System. This system consists of a 24-well multiwell insert plate, and is used to study the movement of cells through a 8 μm-pore size porous fluorescence blocking PET membrane. The light blocking membrane allows the detection of only those cells which migrated through the membrane and attached to the bottom side of the insert.

The bottom side of the inserts is coated with 1.5% gelatine for 2 hours at 37° C. and washed once with PBS. The 24 transwell-insert is placed into a 24-well plate containing 600 μl basal medium+0,1% BSA and supplemented with different growth factors and/or inhibitors to be tested.

Endothelial cells (passage 3-5) are grown in complete growth medium to 70-80% confluency. The cells are harvested by trypsinizing and 30'000 cells in 100% basal medium+0.1% BSA, supplemented or not with inhibitor are added into the insert (upper chamber). The chemoattractant (growth factor) is added only to the lower chamber to stimulate cells to migrate through the pores by establishing a gradient, the different inhibitors are added in both chambers to ensure an equal concentration.

The plates are incubated for 2-5 h at 37° C. Migrated cells located on the lower side of the membranes are fixed with 4% FA for 10 minutes at room temperature, rinsed with PBS, and nuclei were post-stained for 15 minutes with Hoechst dye 33342. The stained cells are automatically counted with the Cellomics ArrayScan™ II cytometer. Each sample is performed in triplicate.

The Cellomics ArrayScan™ II cytometer (Cellomics, Inc., Pittsburgh, Pa.) is an automated fluorescent imaging microscope which is used to scan 16 fields (350 μm in width) per well and to count the cells in each field. Data are analyzed with Microsoft Excel and expressed as the mean-number of migrated cells ±SEM.

Cells transfected with siRNA are used 24 h after transfection.

### Example 19

### Endothelial Cell-Smooth Muscle Cell Coculture Assay (Sprout Formation) for GPR4

Primary human dermal microvascular endothelial cells (HDMECs) and primary human umbilical vein endothelial cells (HUVECs) are purchased from PromoCell, Heidelberg, Germany.

HDMECs are grown in Endothelial Cell Basal Medium MV supplemented with SupplementPack/Endothelial Cell Growth Medium MV and 5% fetal calf serum (FCS), HUVECs are grown in Endothelial Cell Basal Medium supplemented with SupplementPack/Endothelial Cell Growth Medium plus 12.5% FCS.

Endothelial Cell Basal medium is used as minimal or starvation medium.

Human pulmonary artery smooth muscle (HPASM) cells are purchased from PromoCell, Heidelberg, Germany and grown in Smooth Muscle Cell Basal Medium 2 supplemented with SupplementPack/Smooth Muscle Cell Growth Medium 2 and 10% FCS All Media and supplements are purchased at Promocell, Heidelberg, Germany.

Sprout formation of endothelial cells on a layer of smooth muscle cells in the presence of specific growth factors such as VEGF (10 ng/ml), bFGF, PDGF or S1P (100 nM) is measured as a fuction of extracellular pH. In addition, similar experiments are carried out with endothelial cells transfected with an siRNA against GPR4 or In the presence of compounds targeting GPR4 in order to elucidate the specific role of GPR4 in this process. Sprout formation of endothelial cells is used as an in vitro readout for angiogenesis. Inhibition of sprout formation of endothelial cells represents one way of reducing angiogenesis.

48-well tissue culture plates are coated with 10% bovine collagen type I in PBS for 5 h at 37° C. HPASM cells (2×10⁴ cells/well) in 500% smooth muscle cell growth medium II are seeded on the collagen layer and allowed to attach for 24 h at 37° C. and 5% CO₂. The growth medium is removed, and HDMECs or HUVECs (5×10³ cells/well) in endothelial cell growth medium are seeded on top of the confluent HPASM cell monolayer. The cells are incubated for 24 h before medium is replaced with minimal medium (basal+1.5% FCS) containing different growth factors and/or compounds. The plates are incubated for 6 days at 37° C. Medium, growth factors, and compounds are renewed after 2-3 days. On day 6 the medium is removed and the cells are fixed with 4% PFA for 5 minutes at room temperature.

Endothelial cell sprouts are visualized by CD31 staining with an anti-human CD31 antibody (Becton Dickinson) followed by a biotinylated goat anti-mouse antibody and Steptavidin-HRP (horse radish peroxidase) (both Southern Biotechnology). Diaminobenzidine (DAB) colour reaction and counterstaining with Diff-Quick followed.

Cells are imaged at 1.25× magnification using an inverted contrast microscope and acquired images are analyzed using Definiens Cellenger software (Definiens AG, Munich, Germany), which calculates the amount of nuclei, length and area of sprouts, and branching.

For coculture experiments with detection of development over time, Dil-Ac-LDL (20 μg/m) is added to the coculture medium and incubated at 37° C. and 5% CO₂ for 4 hours. The Dil-Ac-LDL staining is renewed every 3 days. Dil-LDL is a fluorescent dye which is taken up specifically by endothelial cells but not by smooth muscle cells and thus allows the detection of endothelial cell sprouts without the need of fixing the cells.

Cells transfected with siRNA are used 24 h after transfection

### Example 20

### In Vivo Angiogenesis Model: Agar Chamber Containing Growth Factors for GPR4

The formation of a highly vascularized tissue around an agar chamber filled with a growth factor is measured in GPR4 k.o mice versus wt mice as well as in wt mice treated with compounds affecting GPR4. This assay is a model for in vivo angiogenesis and is used to characterize anti-angiogenic agents.

A Teflon chamber perforated with 80 regularly spaced 0.8 mm holes and filled under sterile conditions with 0.8% agar plus 20 U/ml heparin with or without a growth factor is implanted s.c. into the flank of the mouse. For subcutaneous implantation, a small skin incision is made at the base of the tail to allow the insertion of an implant trocar. The chamber is implanted under aseptic conditions through the small incision onto the back of the animal. The skin incision is closed by wound clips. After 4 days a vascular tissue forms around the chamber if a growth factor is added into the agar such as VEGF (3 μg/ml) and bFGF (0.3 μg/ml) and S1P (5 mM/ml). On the 4th day after implantation, animals are sacrificed using CO₂. Chambers are then taken out of the animal and the vascularized fibrous tissue formed around each chamber is carefully removed and analysed using different parameters such as weight, haemoglobin and Tie2 protein content. Alternatively the tissue is snapfrozen and processed for histology. The amount of hemoglobin reflects a combination of blood volume and haemorrhages. In contrast, Tie2 is specifically expressed only on endothelial cells, thus Tie2 protein levels can be used as a measure of vascularisation of a certain tissue.

The wet weight of the fibrous tissue that grows around the implant is measured immediately after removal. The whole tissue is then homogenised for 1 min at 24000 rpm (Ultra Turrax T25) after addition of 2 ml of RIPA buffer. The homogenate is centrifuged for 1 hour at 7000 rpm and the supernatant filtered using a 0.45 μm GHP syringe to avoid fat contamination. The amount of haemoglobin and Tie-2 protein is measured in the supernatant. Haemoglobin content is measured by spectrophotometric analysis at 540 nm using the Drabkin reagent kit (Sigma haemoglobin #525). An aliquot of the filtrate (100 μl) is added to 0.9 ml of Drabkin's solution and the mixture incubated for at least 15 minutes at room temperature. The absorbance at 540 nm of the mixture is proportional to the hemoglobin concentration. The hemoglobin measurements are then converted to a blood volume measurement (μl) using a calibration curve previously obtained with whole blood samples of different volumes from a donor mouse. Tie2 protein levels are measured by ELISA: Nunc Maxsisorb 96-well plates are coated over night at 4° C. with 0.1 ml per well of anti-Tie-2 AB33 capture antibody (2 mg/ml, UBI, #05-584). Wells are washed three times and blocked by incubation with 3% Top-Block (Juro #TB232010) 2 h under shaking (300 rpm) at room temperature. Wells are washed again and protein lysates (0.1-0.5 mg in a total volume of 0.2 ml) are added and incubated for 2 h at room temperature. After washing, a complex of Tie2 detection antibody (R&D #AF762, 0.5 μg/ml) and a anti-goat-alkaline phosphates conjugate (diluted 1:6000, SIGMA#A-8062) in PBS-T+0.1% Top-Block is applied for 1 h at room temperature. After washing, Tie-2 antibody complexes are detected by incubating with p-Nitrophenyl phosphate (SIGMA#N-2270, tablets) and absorbance at 405 nm is read. Recombinant human extracellular domain of Tie-2 fused to the constant region of human IgG1 (sTie-2Fc) dissolved in RIPA buffer is used as standard in a concentration range from 0.1 ng-300 ng/well.

### Example 21

### Orthotopic Metastatic Model of 4T1 Mouse Mammary Tumor for GPR4

The growth of orthotopic syngeneic mammary tumors and its metastases is measured in GPR4 k.o mice versus wt mice as well as in wt mice treated with compounds affecting GPR4. Angiogenesis is crucial for tumor growth and metastasis formation.

Tumor cells are inoculated as described by Michigami T et al in Breast Cancer Res Treat. 2002 October;75(3):24958

The 4T1 mouse mammary tumor cells (1×10⁶/0.1 ml PBS) are inoculated orthotopically into the right mammary fat pad of syngeneic female Balb/c mice (6-8 weeks old). Tumors begin to form 7-10 days after cell inoculation. In extensive time course experiments, histological examination revealed that pulmonary and bone metastases begins to develop 2 and 3 weeks after cell inoculation, respectively. Experiments are terminated after 3 weeks and tumors are weighed. Tissues are snapfrozen and either processed for histology or lysed in RIPA buffer for ELISA and Westernblot analysis.

### Example 22

### Orthotopic Metastatic Model of B16-BL6 Mouse Melanoma for GPR4

The growth of orthotopic syngeneic melanoma and its metastases is measured in GPR4 k.o mice versus wt mice as well as in wt mice treated with compounds affecting GPR4. Angiogenesis is crucial for tumor growth and metastasis formation.

B16-BL6 mouse melanoma cells are grown to overconfluency and 5×10⁴ cells in 1 μl HBSS are inoculated orthotopically intradermally (i.d.) Into the skin of the ear of syngeneic female C57/BL6 mice (6-8 weeks old). Injections are performed under a stereotactic microscope and cells are injected at the periphery of the ear between two blood vessels. Tumors begin to form 7-10 days after cell inoculation. Around day 10 metastases in the cranial lymphnode form in 100% of the animals, whereas approximately 30% of the animals had lung metastases after 3 weeks. Experiments are terminated after 3 weeks. Primary tumor size is quantified by imaging whereas lymphnode metastases are weighed. Tissues are snapfrozen and either processed for histology or lysed in RIPA buffer for ELISA and Westernblot analysis.

### Example 24

### Mouse Antigen-Induced Arthritis Model for GPR4

An antigen induced arthritis model is performed in GPR4 k.o mice versus wt mice as well as in wt mice treated with compounds affecting GPR4. Angiogenesis plays an important role in rheumatoid arthritis.

The assay will be performed as previously described by Grosios K et al, Inflamm Res. 2004 April;53(4):133-42. Female mice are sensitised i.d. on the back at two sites to methylated bovine serum albumin (mBSA—Fluka Chemie AG) homogenised 1:1 with complete Freund's adjuvant on days −21 and −14 (0.1 ml containing 1 mg/ml mBSA). On day 0, the right knee receives 10 μl of 10 mg/ml mBSA in 5% glucose solution (antigen injected knee), while the left knee received 10 μl of 5% glucose solution alone (vehicle injected knee). The diameters of the left and right knees are then measured using callipers immediately after the intra-articular injections and again on days 2, 4, 7, 9, 11 and 14. Treatments are administered daily. Right knee swelling was calculated as a ratio of left knee swelling, and the R/L knee swelling ratio plotted against time to give Area Under the Curve (AUC) graphs for control and treatment groups. The percentage inhibition of the individual treatment group AUCs are calculated vs the control group AUC (0% inhibition) using an Excel spreadsheet. On day 14, the mice are killed by CO₂ inhalation and the right and left knees removed and processed for histological analysis.

Knees are processed for undecalcified histology using a Histodurplastic embedding method (Leica AG, Germany). Sections (5 μm) from both the control and arthritic knees were cut on a RM 2165 rotation microtome (Leica AG, Germany). After Giemsa staining, according to standard protocols, the slides are number coded as left knee/right knee pairs from each animal and read in a blinded fashion.

### Example 25

### Rethinopathy of Prematurity Model (ROP)—GPR4

A hypoxia-induced retinopathy model is performed in GPR4 k.o mice versus wt mice as well as in wt mice treated with compounds affecting GPR4. In this model hypoxia is the main driving force inducing angiogenesis in the retina.

The assay is performed as described by Reynolds L E et al, Nat. Med. 2002 January;8(1):27-34 and Wilkinson-Berka J L et al in Invest Ophthalmol Vis Sci. 2003 March;44(3):974-9. ROP is induced in C57BL/6 mice by placing 7-day-old pups with their mother in sealed chambers, containing 75%±5% O₂ and 2% CO₂, using medical grade O₂ and industrial grade air. Gas levels in the chamber are monitored twice daily with a gas analyzer (Model ML 205; AD Instruments, Pty., Ltd., Castle Hill, New South Wales, Australia) and chart recorder (Chart, ver. 3.5, on the MacLab/2E System; AD Instruments, Pty., Ltd.). An airflow rate of approximately 2.5 L/min assists in maintaining adequate levels of metabolically produced CO₂ and decreases in O₂ tension. Mice remain in the chamber for 5 days (hyperoxic period, postnatal day P7-P12) and were then housed in room air for a further 5 days (hypoxia-induced angiogenesis, P12-P17). Mice are perfused with Dextran-FITC to highlight the vessels and retinas are dissected and analysed as wholemounts or by histology.

### Example 26

### Cloning of TDAG8 and Stable Cell Line Generation

Human TDAG8 is amplified from genomic DNA using the following primers: forward, 5′-GACTTCTCTGTTTACTTTCTA-3′; reverse, 5′-GTTCTACTCAAGGACCTCTA-3′. The PCR reaction mixture contains 0.2 mM dNTPs, 1×PCR buffer containing 1.5 mM MgCl2, 0.5 Units Taq/Tgo DNA polymerase mix (Roche), 40 pmol each primer and sterile water in a total volume of 20 μl. The template for this reaction is human genomic DNA (Promega, Wallisellen, Switzerland). PCR is performed using a Biometra T3 Thermocycler using the following cycling conditions: Denaturation at 95° C. for 2 min followed by 38 cycles of denaturation at 95° C. for 30 s, annealing at 60° C. for 45 s, and extension at 72° C. for 1 min. A final extension at 72° C. is performed for 7 min.

The PCR product is cloned into the topo PCR4 vector (Invitrogen, Basel, Switzerland) and sequenced. The cDNA is then re-amplified from this construct using the following primers:

Forward: 5′-TCCGGAATTCGCCACCATGAACAGCACATGTATT-3′

Reverse: 5′-GATCCGCTCGAGCTCAAGGACCTCTAATTC-3′

in order to introduce an EcoRI restriction site and a kozak sequence at the 5′ end of the coding sequence and an XhoI restriction site at the 3′ end, just before the stop codon. The cDNA is then subcloned into the expression vector pcDNA3.1/myc-His as a fusion protein with the myc tag.

Stable cell lines expressing TDAG8 are generated in either CCL39 cells or in CCL39 CRE-luc cells which stably express a cAMP-dependent luciferase reporter to detect changes in cAMP in response to ligand. Stable cell lines are generated In the above cell lines by transfecting the TDAG8 cDNA expression construct using Effectene (Qiagen, Basel, Switzerland) according to the manufacturers protocol. Transfected cells are selected in the presence of 400 μg/ml of G418. After 3 weeks of antibiotic selection, individual clones are picked and expanded for further analysis.

### Example 27

### TDAG8 is a Proton-Sensing G protein-Coupled Receptor Activating cAMP Formation

cAMP formation assay: Confluent cell cultures grown in 24 well plates are labelled with [³H]adenine (100 MBq/ml; Amersham, Dübendorf, Switzerland) for 4 h in serum-free DMEM medium. Cells are then incubated at 37° C. in buffered salt solution as described above for the IP assays (Example 2). Where indicated, the phosphodiesterase inhibitor isobutylmethylxanthine (IBMX, 1 mM) is added to allow accumulation of cAMP. Incubation time is 15 minutes. Cells are then extracted with ice-cold trichloroacetic acid and cAMP separated from free adenine and ATP using batch column chromatography (Salomon Y, 1979, Adv. Cycl Nucleot Res, 10:35-55).

Result: Expression studies and cAMP formation assays as described above, show that this receptor indeed responds to pH shifts in a very similar range as OGR1. Psychosine, which is reported to be a ligand for TDAG8 (Im D S, et al., 2001, J Cell Biol, 153:429-434), does not activate cAMP formation on its own. Half-maximal activation of cAMP formation by TDAG8 in CCL39 cells stably expressing the receptor occurs at pH 7.15+/−0.02 (mean+/−sem; N=6).

### Example 28

### Screening Assay for Agonists or Antagonists of TDAG8

Cells co-expressing TDAG8 and a cAMP luciferase reporter, as described in Example 8, are plated at 10-20,000 cells per well in white 96 well plates. The day before performing the assay the medium is changed for serum-free DMEM and the cells are incubated overnight at 37° C. in the presence of 5% CO₂. Cells are washed once with 100 μl HBS buffer (130 mM NaCl, 0.9 mM NaH₂PO₄, 0.8 mM MgSO₄, 1.8 mM CaCl₂, 5.4 mM KCl, 25 mM glucose, 20 mM HEPES) at the appropriate pH. 100 μl of the appropriate HBS buffer containing the to be tested compound (e.g. from any in-house or commercially available compound library) are then added on the cells. Plates are incubated 4-5 h at 37° C. in a non-CO₂ incubator. After 4-5 h buffer is aspirated from the cells and cells are washed once with 100 μl of PBS. Cells are lysed by addition of 15 μl lysis buffer (5 mM tris-phosphate, 4 mM DTT, 0.4 mM EDTA, 2% glycerol, 0.2% triton X-100), shaking for 1 minute and incubation at room temperature for 20 minutes. Measurement of the light signal upon automatic addition of 50 μl substrate buffer (E1483, Promega, Wallisellen, Switzerland) is performed on a Labsystems Luminoskan RS (BioConcept, Allschwill) with simultaneous substrate dispensing and signal measurement. Agonists in the assay produce an increase in luminescence whereas antagonists produce a decrease in the luminescence. E.g. antagonists are characterised in the assay by a decrease in luminescence signal compared to the no compound control and e.g. in acidic pH buffer at pH 6.8 or pH 7.0 or at half-maximal activation of human TDAG8. Half-maximal activation of luciferase induction by human TDAG8 in CCL39 CRE-luc cells stably expressing the receptor occurs at pH 7.28+/−0.02 (mean+/−sem; N=7).

### Example 29

### Expression of TDAG8 in Osteoclasts and Osteoclast PRECURSOR Cells

Human primary peripheral blood mononuclear cells (PBMNCs, consisting of subsets of monocytes, lymphocytes and other blood cell types) are cultivated in MEMalpha medium suplemented with 10% fetal calf serum. To induce osteoclastic differentiation, some cultures are supplemented with a cytokine cocktail containing M-CSF (25 ng/ml, R&D Systems, Abingdon, UK), RANK ligand (50 ng/ml, Insight Biotechnology, Wembley, UK), TGFbeta1 (5 ng/ml, R&D Systems, Abingdon, UK), dexamethasone (1 microM, Sigma, Buchs, Switzerland). Mature osteoclasts are observed after 17 days of treatment.

RNA is prepared from cultures treated or not with the cytokine cocktail using RNAeasy (Qiagen, Basel, Switzerland). RNA is DNAse-treated and reverse-transcribed using Superscript II (Life technologies/Invitrogen, Basel, Switzerland). Parallel PCR reactions for TDAG8 and glyceraldehydes-3-phosphate dehydrogenase (GDPH) are set up with Expand High Fidelity Taq (Roche) using the following temperature cycling protocol: 30 sec denaturation at 94° C., 45 sec annealing at 56° C. (TDAG8) or 55° C. (GPDH), 50 sec extension at 72° C.; 36 cycles for TDAG8, 30 cycles for GPDH. GPDH is measured as Internal standard for mRNA quantity. For TDAG8, PCR reaction products are cloned and verified by sequencing. The following primers are used: TDAG8 forward: 5′-AAMCTACTTGTCAGCATCACA-3′, reverse: 5′-GTTCTACTCAAGGACCTCTA-3′; GPDH forward: 5′-TTAGCACCCCTGGCCAAGG-3′, reverse: 5′-CTTACTCCTTGGAGGCCATG-3′.

Results: Strong expression of TDAG8 is observed both in PBMNCs and fully differentiated osteoclasts.

### Example 30

### Osteoclast Differentiation and Activity Assay (Functional Assay for TDAG8)

Human primary peripheral blood mononuclear cells (PBMNCs) are cultivated in MEMalpha medium suplemented with 10% fetal calf serum. To induce osteoclastic differentiation, cultures are supplemented with a cytokine cocktail containing M-CSF (25 ng/ml, R&D Systems, Abingdon, UK), RANK ligand (50 ng/ml, Insight Biotechnology, Wembley, UK), TGFbeta1 (5 ng/ml, R&D Systems, Abingdon, UK), dexamethasone (1 microM, Sigma, Buchs, Switzerland). To study pH effects on osteoclast differentiation, cells are maintained in medium of variable pH during differentiation, and mature osteoclasts counted under the microscope following staining for tartrate-resistant acid phosphatase.

To study pH effects on osteoclast activity, PBMNCs are seeded onto bovine bone slices and cultured in the presence of cytokine cocktail as described above under standard conditions until day 14. Cultures are then shifted to medium of variable pH, and after three further days osteoclast activity is assessed by measuring area of resorbed bone and concentration of released collagen fragments. In addition, human TDAG8 antagonists (e.g. obtained by the screening assay of example 17) may revert the observed agonistic effect of a neutral environment such as pH 6.8 or 7.0.

### Example 31

### Pairwise Sequence Alignments

Sequences are aligned using the GCG programme ‘Gap’ (Devereux J, et al., 1984, Nucl Acids Res, 12:387-395.)

Protein Sequences used with Refseq database accession numbers

OGR1 NP_{—}003476

GPR4 NP_{—}005273

TDAG8 NP_{—}003599

Human OGR-1 alignment with human GPR4: Percent Similarity: 50.838 Percent Identity: 45.251

Human OGR-1 alignment with human TDAG8: Percent Similarity: 45.706 Percent Identity: 34.663

Human GPR4 alignment with human TDAG8: Percent Similarity: 46.061 Percent Identity: 36.970

1. The use of an isolated proton-sensing GPCR polypeptide in the development of a medicament for disease and medical conditions in which proton homeostasis is imbalanced.

2. The use of an isolated proton-sensing GPCR polypeptide in the development of a medicament for disease and medical conditions in which proton homeostasis is imbalanced; said polypeptide is selected from one of the groups consisting of:
(a) an isolated polypeptide encoded by a polynucleotide comprising the polynucleotide sequence of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPM (accession number: NM_{—}005282), mouse GPR4 (accession number: NM_{—}175668), human TDAG8 (accession number: NM_{—}003608) and mouse TDAG8 (accession number: NM_{—}008152);
(b) an isolated proton sensing GPCR polypeptide comprising a polypeptide sequence having at least 20% to the polypeptide sequence of SEQ ID NO: 1;
(c) an isolated proton sensing GPCR polypeptide comprising a polypeptide sequence having at least 20% to the polypeptide sequence of SEQ ID NO: 3;
(d) an isolated proton sensing GPCR polypeptide comprising a polypeptide sequence having at least 20% identity to the polypeptide sequence of SEQ ID NO: 4;
(e) an isolated polypeptide comprising the polypeptide sequence of SEQ ID NO: 1, SEQ ID NO: 3 or SEQ ID NO: 4;
(f) an isolated proton sensing GPCR polypeptide having at least 20% identity to the polypeptide sequence of SEQ ID NO: 1;
(g) an isolated proton sensing GPCR polypeptide having at least 20% identity to the polypeptide sequence of SEQ ID NO: 3;
(h) an isolated proton sensing GPCR polypeptide having at least 20% identity to the polypeptide sequence of SEQ ID NO: 4;
(i) the polypeptide sequences of SEQ ID NO: 1, SEQ ID NO: 3, or SEQ ID NO: 4; and
(j) polypeptides in (a) to (i) which show a pH dependent Inositol phosphate formation in CCL39 hamster fibroblast cells or a pH dependent signal in the cAMP luciferase reporter assay in CHOK1 CRE-luc cells or CCL39 CRE-luc cells.

3. The use of the isolated polypeptide as claimed in claim 1 or 2, said polypeptide comprising the polypeptide sequence of SEQ ID NO: 1, SEQ ID NO: 3 or SEQ ID NO: 4.

4. The use of the isolated polypeptide as claimed in claim 1, said polypeptide consisting of the polypeptide sequence of SEQ ID NO: 1, SEQ ID NO: 3 or SEQ ID NO: 4.

5. The use of an isolated polynucleotide in the development of a medicament for the prevention and treatment of diseases and medical conditions in which proton homeostasis is imbalanced; said polynucleotide is selected from one of the groups consisting of:
(a) an isolated polynucleotide comprising the polynucleotide sequence of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number: NM_{—}008152);
(b) an isolated polynucleotide encoding a proton sensing GPCR polypeptide sequence having at least 20% identity to the polypeptide sequence of SEQ ID NO: 1;
(c) an isolated polynucleotide encoding a proton sensing GPRC polypeptide sequence having at least 20% identity to the polypeptide sequence of SEQ ID NO: 3;
(d) an isolated polynucleotide encoding a proton sensing GPRC polypeptide sequence having at least 20% identity to the polypeptide sequence of SEQ ID NO: 4;
(f) an isolated polynucleotide comprising the polynucleotide sequence of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number: NM_{—}008152);
(g) the polynucleotide sequences of human OGR1 (accession number: NM_{—}003485.1), rat OGR1 (accession number: XM_{—}234483), mouse OGR1 (accession number: NM_{—}175493), bovine OGR1 (accession number: NM_{—}174329), preferably human OGR1 (accession number: NM_{—}003485.1), human GPR4 (accession number: NM_{—}005282), mouse GPR4 (accession number: NM_{—}175668), human TDAG8 (accession number: NM_{—}003608) and mouse TDAG8 (accession number NM_{—}008152); and
(h) polynucleotides in (a) to (g) which encode for a polypeptide that show a phi dependent Inositol phosphate formation in CCL39 hamster fibroblast cells or a pH dependent signal in the cAMP luciferase reporter assay in CHOK1 CRE-luc cells or CCL39 CRE-luc cells.

6. The use of an antibody, which specifically binds to a polypeptide of the claim 1, for the manufacture of a medicament for the prevention and/or treatment of diseases and medical conditions in which proton homeostasis is imbalanced;
a pharmaceutical composition comprising an antibody for the prevention and/or treatment of diseases and medical conditions in which proton homeostasis is imbalances, said antibody specifically binds to a polypeptide of the claim 1; or
a method of prevention and/or treatment of diseases and medical conditions in which proton homeostasis is imbalanced comprising administering to a subject in need of such prevention and/or treatment an effective amount of an antibody, said antibody specifically binds to a polypeptide of the claim 1.

7. A method for screening to identify compounds that antagonize the proton-sensing activity of the polypeptides according to claim 1.

8. A method for screening to identify compounds that agonize the proton-sensing activity of the polypeptides according to claim 1.

9. A method for screening to identify compounds that stimulate or inhibit the function or expression level of the polypeptides of claim 1 comprising a method selected from the group consisting of:
(a) measuring or, detecting, quantitatively or qualitatively, the binding of a candidate compound to the polypeptide (or to the cells or membranes expressing the polypeptide) or a fusion protein thereof by means of a label directly or indirectly associated with the candidate compound;
(b) measuring the competition of binding of a candidate compound to the polypeptide (or to the cells or membranes expressing the polypeptide) or a fusion protein thereof in the presence of a labelled competitor;
(c) testing whether the candidate compound results in a signal generated by activation or inhibition of the polypeptide, using detection systems appropriate to the cells or cell membranes expressing the polypeptide; or
(d) detecting the effect of a candidate compound on the production of mRNA encoding said polypeptide or said polypeptide in cells, using for instance, an ELISA assay.

10. A method of prevention and/or treatment of diseases and medical conditions in which proton homeostasis is imbalanced comprising administering to a subject in need of such prevention and/or treatment an effective amount of an antagonist obtainable from the method of claim 7.

11. A method of prevention and/or treatment of diseases and medical conditions in which proton homeostasis is imbalanced comprising administering to a subject in need of such prevention and/or treatment an effective amount of an agonist obtainable from the method of claim 8.

12. A pharmaceutical composition for the prevention and/or treatment of diseases and medical conditions in which proton homeostasis is imbalanced comprising an antagonist obtainable from the method of claim 7.

13. A pharmaceutical composition for the prevention and/or treatment of diseases and medical conditions in which proton homeostasis is imbalanced comprising an agonist obtainable from the method of claim 8.

14. A diagnostic kit comprising an antibody against a polypeptide according to claim 1.

15. A diagnostic kit comprising a pharmaceutical preparation for the prevention and/or treatment of diseases and medical conditions in which proton homeostasis is imbalances, said pharmaceutical preparation comprising an antibody against a polypeptide according to claim 1.


SEQUENCE LISTING (6 sequences)

SEQ ID NO: 1 - protein, 365 aa, H. sapiens
MGNITADNSSMSCTIDHITHQTLAPVVYVTVLVVGFPANCLSLYFGYLQIKARNELGVYLCNLTVADLFYICSLPFWLQYVLQHDNWSHGDLSCQVCGILLYENIYISVGFLCCISVDRYLAVAHPFRFHQFRTLKAAVGVSVVIWAKELLTSIYFLMHEEVIEDENQHRVCFEHYPIQAWQRAINYYRFLVGFLFPICLLLASYQGILRAVRRSHGTQKSRKDQIQRLVLSTVVIFLACFLPYHVLLLVRSVWEASCDFAKGVFNAYHFSLLLTSFNCVADPVLYCFVSETTHRDLARLRGACLAFLTCSRTGRAREAYPLGAPEASGKSGAQGEEPELLTKLHPAFQTPNSPGSGGFPTGRLA

SEQ ID NO: 2 - protein, 16 aa, Artificial Sequence
  Peptide used for antibody production
CFVSETTHRDLARLRG

SEQ ID NO: 3 - protein, 362 aa, H. sapiens
MGNHTWEGCHVDSRVDHLHPPSLYIFVIGVGLPTNCLALWAAYRQVQQRNQLGVYLMNLSIADLLYICTLPLWVDYFLHHDNWIHGPGSCKLPGFIFYTNIYISIAFLCCISVDRYLAVAHPLRFARLRRVKTAVAVSSVVWATELGANSAPLFHDELFRDRYNHTFCFEKFPMEGWVAWMNLYRVFVGFLFPWALMLLSYRGILRAVRGSVSTERQEKAKIKRLALSLIAIVLVCFAPYHVLLLSRSAIYLGRPWDCGFEERVFSAYHSSLAFTSLNCVADPILYCLVNEGARSDVAKALHNLLRFLASDKPQEMANASLTLETPLTSKRNSTAKAMTGSWAATPPSEGDQVQLKMLPPAQ

SEQ ID NO: 4 - protein, 337 aa, H. sapiens
MNSTCIEEQHDLDHYLFPIVYIFVIIVSIPANIGSLCVSFLQPKKESELGIYLFSLSLSDLLYALTLPLWIDYTWNKDNTTFSPALCKGSAFLMYMLFYSSTAFLTCIAVDRYLAVVYPLKFFFLRTRRIALMVSLSIWILETIFNAVMLWEDETVVEYCDAEKSNFTLCYDKYPLEKWEINLNLFRTCTGYAIPLVTILICNRKVYQAVRHNKATENKEKKRIIKLLVSITVTFVLCFTPFHVMLLIRCILEHAVNFEDHSNSGKRTYTMYRITVALTSLNCVADPILYCFVTETGRYDMWNILKFCTGRCNTSQRQRKRILSVSTKDTMELEVLE

SEQ ID NO: 5 - protein, 19 aa, Artificial Sequence
  Peptide used for antibody production
RSDVAKALHNLLRFLASDK

SEQ ID NO: 6 - protein, 20 aa, Artificial Sequence
  Peptide used for antibody production
DELFRDRYNHTFCFEKFPME